(19) United States
(12) Patent Application Publication
(10) Pub. No.: US 2026/0234217 A1
(43) Pub. Date: Aug. 13, 2026

(54) USE OF BMP RECEPTOR ALK1 INHIBITORS IN OSTEOARTHRITIS THERAPIES

(71) Applicant: The Regents of the University of California, Oakland, CA (US)

(72) Inventors: Weiguang Wang, Culver City, CA (US); Karen Lyons, Encino, CA (US)

(73) Assignee: The Regents of the University of California, Oakland, CA (US)

(21) Appl. No.: 19/156,771

(22) PCT Filed: Feb. 28, 2024
(86) PCT No.: PCT/US2024/017657

§ 371 Date: Aug. 14, 2025

Related U.S. Application Data
(60) Provisional application No. 63/487,749, filed on Mar. 1, 2023.
(60) Provisional application No. 63/513,246, filed on Jul. 12, 2023.

(51) Int. Cl. C07K 14/71; A61K 9/00; A61K 31/496; A61K 38/18; A61P 19/02

(52) U.S. Cl. CPC C07K 14/71; A61K 9/0019; A61K 31/496; A61K 38/18; A61P 19/02; C07K 2319/30; C12Y 207/11001

(57) ABSTRACT

Osteoarthritis (OA) is a common clinical problem that constitutes an enormous global healthcare burden. Effective non-surgical treatment approaches are a major unmet clinical need. This disclosure describes the use of ALK1-Fc fusion proteins, growth differentiation factor 11 (GDF11) polypeptides and/or small molecules such as LDN-214117 in methods for OA treatment.

### CROSS REFERENCE TO RELATED APPLICATIONS

This application claims the benefit under 35 U.S.C. Section 119(e) of co-pending and commonly-assigned U.S. Provisional Patent Applications No. 63/487,749, filed Mar. 1, 2023, entitled “A NEW INDICATION FOR BMP RECEPTOR ALK1 INHIBITORS AS AN OSTEOARTHRITIS THERAPY” and U.S. Provisional Patent Applications No. 63/513,246, filed Jul. 12, 2023, entitled “A NEW INDICATION FOR BMP RECEPTOR ALK1 INHIBITORS AS AN OSTEOARTHRITIS THERAPY, which applications are incorporated by reference herein.

### STATEMENT AS TO RIGHTS TO INVENTIONS MADE UNDER FEDERALLY SPONSORED RESEARCH AND DEVELOPMENT

This invention was made with government support under Grant Number AR073793, awarded by the National Institutes of Health. The government has certain rights in the invention.

### TECHNICAL FIELD

The fields of the invention include medicine and pharmacology.

### BACKGROUND OF THE INVENTION

Osteoarthritis (OA) is the most common form of arthritis, affecting millions of people worldwide and more than 32.5 million Americans [1]. It occurs when the protective cartilage that cushions the ends of bones wears down. This is accompanied by the formation of heterotopic bone (osteophytes) in the joint space. OA can be induced by physical injury to a joint (PTOA) or by normal aging. Although OA can damage any joint, the disorder most commonly affects joints in hands, knees, hips and spine. Besides the breakdown of cartilage, OA affects the entire joint. It causes osteophyte formation on the surfaces of joints, increases abnormal ossification in the underlying bone, leads to deterioration of the connective tissues that hold the joint together and comprise attachments of muscle to bone (e.g., tendons and ligaments), and induces heterotopic ossification around the joint tissues. There are currently no disease-modifying drugs for treatment of OA. At present, a damaged joint can be surgically replaced with a metal, plastic or ceramic one, but this is a painful process that takes up to 3 months for full recovery and is not a permanent solution. In fact, OA is one of the most expensive conditions to treat when joint replacement is required [2]. As such, OA constitutes an enormous musculoskeletal healthcare challenge and an effective pharmaceutical agent is urgently required to fulfill the major unmet OA therapy need.

Crosstalk between TGFβ and BMP signaling pathways plays an important role in maintaining the health of cartilage and bone. Disruption of the balance between TGFβ vs. BMP signaling is correlated with severe OA in humans [3]. Particularly, Davidson's paper reported that the expression level of TGFβ type I receptor ALK5 is positively correlated with the expression of the healthy cartilage matrix components collagen type II and aggrecan, while the expression level of BMP type I receptor ALK1 is corelated with the expression of MMP-13, an enzyme that degrades cartilage and serves as a marker gene for OA. In addition, this publication reported that the expression level of ALK5 is decreased and the ALK1/ALK5 ratio increases with OA in the destabilization of the medial meniscus (DMM) mouse model of PTOA. However, it has not been determined whether the change in expression of ALK5 or ALK1 causes OA in vivo, whether there is a direct role for ALK1 in cartilage in vivo, or whether modifying the ALK5/ALK1 ratio could alter the course of OA.

There is a need in this technology for a better understanding of the biological mechanisms associated with osteoarthritis, and for additional agents for the treatment of osteoarthritis and methods for using such agents.

### SUMMARY OF THE INVENTION

The present invention is based in part upon the inventors' studies of a regulatory system of receptor crosstalk between ALK5 and ALK1 in cartilage maintenance, and the role of this system in osteoarthritis. The disclosure presented herein shows the regulation of ALK1 mediated signaling in maintain articular cartilage integrity, osteophyte formation, bone ossification, and heterotopic ossification in vivo. Building upon these studies, the invention disclosed herein describes the use of selected ALK1 antagonists as protective agents for OA therapy, articular cartilage protection, cartilage regeneration, prevention of osteophyte formation and inhibition of abnormal bone ossification and heterotopic bone formation in mammals. As discussed below, the invention disclosed herein further describes the use of selected ALK1 antagonists as pain mitigating agents in OA therapies.

Embodiments of the invention provide antagonists of the receptor ALK1 or ActRIIB or the ligands BMP9 and BMP10, and methods for their use in OA therapies. Illustrative antagonists include ALK1-Fc fusion proteins, growth differentiation factor 11 (GDF11) polypeptides, and small molecules such as LDN-214117. In certain aspects, the invention provides such antagonists for the treatment of cartilage degradation and abnormal bone ossification, particularly post-traumatic osteoarthritis, osteoarthritis, rheumatoid arthritis, and disorders associated with pathological ossification in the joint, tendon, ligament, muscle, skin, vessels and vascularized tissues.

Embodiments of the invention include methods of promoting cartilage preservation, repair, and/or regeneration in a mammal (e.g. a human diagnosed with osteoarthritis (OA)), the methods comprising administering to the mammal a therapeutically effective amount of at least one agent selected from an ALK1-Fc fusion protein, a GDF11 polypeptide, and a small molecule such as LDN-214117, such that cartilage preservation, repair, and/or regeneration is promoted. Typically in these embodiments, the mammal is administered an amount of agent selected to be sufficient to inhibit heterotopic ossification, osteophyte formation, and/or destruction of articular cartilage in vivo. Related embodiments of the invention include methods of modulating the physiology of an articular cartilage, the method comprising combining the articular cartilage with amounts of an ALK1-Fc fusion protein, a GDF11 polypeptide, and/or LDN-214117 selected to be sufficient to inhibit fibrocartilage formation; and/or inhibit osteophyte formation; thereby modulating the physiology of articular cartilage. Typically in these methods, the articular cartilage is disposed in a human diagnosed with osteoarthritis (OA). Optionally in these methods, the articular cartilage can be combined with ALK1-Fc by intra-articular injection of the ALK1-Fc (e.g. in amounts of 300 ng-500 ng per dose per week for at least 1-8 weeks).

In certain aspects, the invention provides methods for promoting an increase in chondrocyte survival in a cartilaginous tissue of a mammal in need thereof, the method comprising administering to the mice an effective amount of an ALK1-Fc protein (optionally with one or more additional agents such as a GDF11 polypeptide or LDN-214117) by injecting intro-cavity in the knee joint one dose of 400 ng/per week for 8 weeks. In certain embodiments, the invention provides methods for inhibiting osteophyte formation in a mammal by administering a plurality of the ALK1 antagonists disclosed herein. In one illustrative embodiment, a method of the invention comprises administering to mice an effective amount (e.g., one dose of 400 ng/per week for 8 weeks) of an ALK1-Fc fusion protein, or an ALK1 and ALK2 kinase inhibitor LDN-214117 (e.g., one dose of 16 ng/per week for 8 weeks). In certain embodiments, the invention provides methods for inhibiting abnormal ossification of joints in a mammal by administering any of the ALK1 antagonists disclosed herein. In certain aspects, the disclosure provides methods for inhibiting heterotopic bone formation in joint, tendon, or ligament in a mammal by administering any of the ALK1 antagonist specifically herein.

As shown for example in the data presented in FIG. 7, it was discovered that there is a significant decrease in pain in DMM mice treated with ALK1-Fc, and also in DMM mice treated with LDN-214117. In this context, embodiments of the invention include methods of mitigating patient pain associated with damage to articular cartilage, the method comprising combining the articular cartilage with amounts of at least one agent selected from ALK1-Fc and LDN-214117 in amounts sufficient to inhibit pain in the patient following trauma to the articular cartilage; and optionally to simultaneously inhibit the degradation of the articular cartilage following trauma to the articular cartilage; and/or inhibit the development and/or progression of osteoarthritis following trauma to the articular cartilage; such that pain associated with damage to articular cartilage is mitigated.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. ALK1 mediates degeneration of articular cartilage in PTOA. (A) Expression by IHC of ALK1 (Red) and DAPI (blue) in medial tibial plateau articular cartilage in: Wild-Type (WT; lacking Col2-Cre) mice that underwent sham surgery (Sham+WT), WT mice that underwent DMM surgery (DMM+WT), and mice with ALK1 knocked out in cartilage (Alk1fx/fx; Col2a1-Cre, referred to as Alk1Col2 hereafter) that underwent DMM surgery (DMM+Alk1Col2) (negative control). (B) Safranin-O/Fast Green (SOFG) Staining of sagittal sections of the knee joint, femur (top) and tibia (bottom) for proteoglycan (cartilage; red) and bone (green) at week 8 in: WT mice that underwent sham surgery (Sham+WT), WT mice that underwent DMM surgery (DMM+WT), and mice with ALK1 knocked out in cartilage via Col2a1-Cre that underwent Sham surgery (Sham+Alk1Col2), Mice with ALK1 knocked out in articular cartilage via Col2-Cre who underwent DMM surgery (DMM+Alk1Col2). (C) Expression by TIC (green) and DAPI (blue) of pSMAD1/5 in medial tibial plateau articular cartilage (D-E) Osteoarthritis Research Society International (OARSI) scoring of articular cartilage damage in the (D) medial tibial plateau and (E) medial femoral condyle in representative sections of each groups at week 8 after surgery. (F) Quantified expression by percent positive cells for pSMAD1/5. All data is shown as mean bar+/−standard deviation *p<0.05, **p<0.01, ***p<0.001, ****p<0.0001. Data analysis performed was two-way ANOVA. Scale in A-C 250 uM.

FIG. 2. Inhibition of ALK1/2 prevents articular cartilage degeneration when administered immediately after DMM surgery. (A) Experimental Outline (B) Safranin-O/Fast Green (SOFG) Staining of sagittal sections of the knee joint, femur (top) and tibia (bottom) with proteoglycan (red) and bone (green) at week 8 after DMM or Sham surgery. Mice were treated with either BMP Kinase inhibitor LDN-214117 (BMP-KI), Ligand Trap targeting ALK1 (ALK1-Fc), or Phosphate Buffered Saline (PBS) after undergoing surgery. Articular cartilage damage is indicated by a dotted line. (C-D) Osteoarthritis Research Society International (OARSI) scoring of articular cartilage damage in the (C) medial tibial plateau and (D) medial femoral condyle in representative sections of each treatment at week 8 after surgery. The data for the sham (WT and PBS) and DMM (WT and PBS) groups in FIGS. 1 and 2 are the same. All data are shown as mean+/−standard deviation *p<0.05, **p<0.01, ***p<0.001, ****p<0.0001. Data analysis was performed with two-way ANOVA. Scale in B 250 uM.

FIG. 3. ALK1/2 kinase inhibitor BMP-KI and ligand-trap ALK1-Fc reduce BMP signaling in AC. All images are of sagittal sections through the medial compartment of 5-mo old mouse knee joints. Expression by IHC (green) and DAPI (blue) of (A) pSMAD1/5, (B) pSMAD2/3, (C) Collagen-X (COL10), and (D) Matrix Metalloproteinase-13 (MMP13) Expression. (E) The percentage of cells positive for pSMAD1/5 is elevated in DMM mice, but reduced in mice treated with BMP-KI and ALK1-Fc. (F) p SMAD2/3 expression is not rescued to levels seen in healthy sham control AC. (G,H) Quantified expression of the percent of cells expressing COL10 (G) or MMP13 (H) demonstrates treatment with ALK1-Fc restores levels of expression to those seen in healthy sham control AC, and treatment with BMP-KI significantly decreases levels of expression in DMM AC when treated immediately after DMM surgery. All data are shown as mean+/−standard deviation *p<0.05, **p<0.01, ***p<0.001, ****p<0.0001. Data analysis was performed using two-way ANOVA with Tukey's Multiple Comparisons Test.

FIG. 4. ALK1/2 inhibitors attenuate articular cartilage degeneration in established OA. (A) Experimental Outline (B) Safranin-O/Fast Green (SOFG) Staining of sagittal sections of the knee joint, femur (top) and tibia (bottom) for proteoglycan (cartilage; red) and bone (green) at week 8 after DMM or Sham surgery. Mice were treated with either BMP Kinase inhibitor LDN-214117 (BMP-KI), Ligand Trap targeting ALK1 (ALK1-Fc), or Phosphate Buffered Saline (PBS) after received surgery (n=5/group). 4-Week DMM mice were sacrificed 4 weeks after surgery and received no treatment (n=6). AC damage indicated by dotted line. (C-D) Osteoarthritis Research Society International (OARSI) scoring of AC damage in the (C) medial tibial plateau and (D) medial femoral condyle in representative sections of each treatment at week 4 or 8 after surgery. All data are shown as mean bar+/−standard deviation *p<0.05, **p<0.01, ***p<0.001, ****p<0.0001. Data analysis performed was two-way ANOVA. Scale 250 uM.

FIG. 5. Delayed administration of ALK1/2 kinase inhibitors maintains proper BMP signaling in AC. All images are of sagittal sections through the medial compartment of 5-mo old mouse knee joints. Expression by IHC (green) and DAPI (blue) of (A) pSMAD1/5, (B) pSMAD2/3, (C) Collagen-X (COL10), and (D) Matrix Metalloproteinase-13 (MMP13) Expression. (E) The percentage of cells positive for pSMAD1/5 is elevated in DMM mice, but reduced in mice treated with BMP-KI and ALK1-Fc. (F) Quantification of percent cells positive for pSMAD2/3 in mice treated with BMP-KI and ALK1-Fc; levels are not rescued to those seen in healthy sham control AC. (G,H) Quantified expression of the percentage of cells expressing COL10 (G) or MMP13 (H). Col10 levels in DMM mice treated with KI and Fc were significantly decreased compared to the PBS group, and MMP13 levels in mice treated with KI and Fc are attenuated to those of healthy sham control AC when treated 1 month after DMM surgery. All data are shown as mean+/−standard deviation *p<0.05, **p<0.01, ***p<0.001, ****p<0.0001. Data analysis was performed using two-way ANOVA with Tukey's Multiple Comparisons Test.

FIG. 6. Inhibiting ALK1 reduces the formation of osteophytes at the joint. (A) Three dimensional representation of the knee joint imaged by uCT of mice at week 8 after Sham or DMM surgery. Mice were treated with either Phosphate Buffered Saline (PBS), BMP Kinase inhibitor LDN-214117 (BMP-KI), or ALK1 Constant Fragment Ligand Trap (ALK1-Fc) after received surgery (n=5/group). Red arrows indicate osteophytes in the DMM joint. The femur is on top, and the tibia is on the bottom. (B) Total osteophyte volume of knee joints of WT or ALK1Col2 mutant mice at week 8 after DMM or Sham surgery. (n=5/group). (C) Total osteophyte volume of knee joints of mice undergoing each treatment at week 8 after DMM or Sham surgery. Mice were treated with either BMP-KI, ALK1-Fc, or PBS immediately after received surgery (n=5/group). (D) Total osteophyte volume of knee joints of mice undergoing each treatment with a 1-month delay after surgery. All data is shown as mean bar+/−standard deviation *p<0.05, **p<0.01, ***p<0.001, ****p<0.0001. Data analysis was performed using two-way ANOVA with Tukey's Multiple Comparisons Test.

FIG. 7. Administration of ALK1/2 kinase inhibitors reduce OA Associated Pain. Von Frey Microfilament pain sensitivity test shows the average 50% paw withdrawal threshold for mice undergoing DMM surgery. Mice underwent pain testing weekly, beginning immediately prior to DMM surgery (Week 0), and continuing for 8 weeks until the date of harvest (Week 8). By week 6, mice undergoing sham surgery and DMM surgery treated with ALK1-Fc show a decreased pain response threshold compared to DMM control mice. By week 8, mice undergoing DMM surgery treated with BMP-KI show a decreased pain response threshold compared to DMM control mice. #, ##, ###denote significant difference (p<0.05, p<0.01, p<0.001) between Sham and DMM+PBS groups respectively. *, ** denote significant difference (p<0.05, p<0.01) between DMM+ALK1-Fc and DMM+PBS groups respectively. \hat{ } denotes significant difference (p<0.05) between DMM+BMP-KI and DMM+PBS groups respectively. Data was analyzed with two-way ANOVA with Tukey's Multiple Comparisons Test.

FIG. 8. scRNA-Seq analysis reveals increased ALK1 expression in human OA cartilage. The dataset was obtained from Chou et al(49). Chondrocytes were harvested from the outer intact lateral (Control) and inner damaged medial (OA) tibial articular cartilages(49). (A-C) Subpopulations of chondrocytes in OA and control cartilage were identified through tSNE projection. It is noted that fibrochondrocytes (FC) and pre-fibrochondrocytes (preFC) are enriched in OA cartilage. (D) ALK1 mRNA expression was specifically increased in OA FC and preFC. (E) ALK2 mRNA expression was enriched in OA FC and preFC, but it was also highly expressed in other populations in both intact and OA cells compared to ALK1. (F) ALK1 expression showed a 32-fold increase, and ALK2 showed a 1.5-fold increase in human OA compared to control articular cartilage.

FIG. 9. The expression of ALK1 is associated with the expression of marker genes for terminally differentiated and fibrotic chondrocytes in human OA. The scRNA-Seq dataset was obtained from Chou et al. (34). mRNA expression levels of genes were normalized using Gapdh levels. Data for SOX9, MMP13, COL10A1, ADAMTS2, COL1A1, IL11, COL1A2, and ID3 were plotted for different subpopulations of chondrocytes and categorized by OA and control cells.

FIG. 10. Genetic deletion or pharmaceutical inhibition of ALK1 has no obvious impact on the adult growth plate maintenance. Sagittal sections of the proximal tibial growth plate were stained with Safranin-O/Fast Green (SOFG) to visualize proteoglycan (red) in the growth plate, subchondral bone (top blue), and trabecular bone (bottom blue) at week 8 after DMM or Sham surgery. Wild-type DMM mice were subjected to one of the following treatments: BMP kinase inhibitor LDN-214117 (DMM+BMP-KI), Ligand Trap targeting ALK1 (DMM+ALK1-Fc), or Phosphate Buffered Saline (DMM+PBS). Alk1^{Col2} mice underwent DMM surgery (DMM+Alk1^{Col2}). WT control mice (lacking Col2a1-Cre) underwent sham surgery and received PBS treatment (Sham+PBS). (n=5/group).

FIG. 11. ALK1 protein is highly expressed in osteophytic chondrocytes in DMM-induced OA mice. Safranin-O/Fast Green (SOFG) staining of sagittal sections of the knee joint femur (A, B, D, E) reveals cartilage proteoglycan (in red) and bone (in green) at week 8 (5 months of age) after DMM surgery in the following scenarios: (A) mice that underwent Sham surgery, and (D) mice that underwent DMM surgery. High-magnification images from the boxed areas in (A) and (D) are shown in (B) and (E). The yellow circle in (E) indicates cartilage accumulation around the perichondrium and osteophyte formation in the DMM femur. In (C) and (F), ALK1 expression by immunohistochemistry (IHC, in red) was detected at a low level in normal articular cartilage (labeled as AC in yellow) (C), but it was enriched in the osteophytic region (yellow circle) in the DMM femur (F). The yellow ‘F’ label indicates that ALK1 expression is associated with increased fibrotic cells and is present on the surface of the osteophyte region. The yellow ‘S’ label indicates that ALK1 expression is observed on the surface of the synovium. Blue staining represents DAPI for cell nuclei.

FIG. 12. Pharmaceutical inhibition of ALK1 has no significant impact on the thickness and volume of subchondral bone plates in DMM-induced OA mice. (A) Safranin-O/Fast Green (SOFG) staining of sagittal sections of the knee joint, including the femur (top) and tibia (bottom), with proteoglycan (in red) and bone (in green) was performed at week 8 (5 months of age) after DMM or Sham surgery. Mice received treatments with either BMP kinase inhibitor LDN-214117 (DMM+BMP-KI), Ligand Trap targeting ALK1 (DMM+ALK1-Fc), or Phosphate Buffered Saline (DMM+PBS) after undergoing DMM surgery. Control mice received sham surgery and PBS treatment (Sham+PBS) (n=5/group). It should be noted that the thickness of the subchondral bone plate beneath the tibia articular cartilage increased in all DMM femurs compared to the Sham tibia. (B and C) Quantification through microCT analysis indicates that BMP-KI and ALK1-Fc have no significant impact on the thickness (B) and total volume (C) of the subchondral plates in DMM-induced OA mice, regardless of the treatment with BMP-KI, ALK1-Fc, or PBS.

FIG. 13. Growth differentiation factor 11 (GDF11, see, e.g., NCBI Reference Sequence: NP_005802.1) competes with BMP9/10 for binding to BMP type II receptors ACVRIIA or ACVRIIB, thereby preventing the association of BMP type I receptor ALK1 with ACVRIIA/B and decreasing BMP signaling. Additionally, GDF11 can increase TGFbeta signaling. We tested this hypothesis by locally injecting GDF11 (50 ng/dose) into the knee joint space at mice with established post-traumatic OA. (A) Experimental Outline (B) Safranin-O/Fast Green (SOFG) Staining of sagittal sections of the knee joint, femur (top) and tibia (bottom) for proteoglycan (cartilage; red) and bone (green) at week 8 after DMM or Sham surgery. Mice were treated with either GDF11, or Phosphate Buffered Saline (PBS) after received surgery (n=5/group). 4-Week DMM mice were sacrificed 4 weeks after surgery and received no treatment (n=6). AC damage indicated by dotted line. (C-D) Osteoarthritis Research Society International (OARSI) scoring of AC damage in the (C) medial tibial plateau and (D) medial femoral condyle in representative sections of each treatment at week 4 or 8 after surgery. All data are shown as mean bar+/−standard deviation *p<0.05. Data analysis performed was one-way ANOVA. Scale 250 uM.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

ALK1 is a receptor for the BMP9 and BMP10 ligands. ALK1 normally complexes with ActRIIA and ActRIIB, but in healthy cartilage, ALK1 is kept inactive by complex formation with ALK5. When ALK5 levels are decreased or ALK1 levels are increased, the levels of ALK1 complexed with ActRIIB increase, resulting in elevated BMP signaling through ALK1. This demonstrates that signaling mediated by ALK1 and ActRIIB and the ligands described above is involved in OA pathology in vivo, and that inhibition of this regulatory system has a potent joint cartilage and bone protection effect. Additionally, this demonstrates that inhibition of the ALK1 regulatory system causes decreased degradation of articular cartilage and inhibits osteophyte formation and abnormal ossification in joint tissues.

Loss of ALK5 in growth plate cartilage causes severe chondrodysplasia and lethality (Wang et al., Proc Natl Acad Sci USA. 2019 Jul. 30; 116(31):15570-15579) [4]. ALK5 is thought to mediate the majority of its effects via the binding of TGFβ ligands to a complex formed by ALK5 and the TGFβ type II receptor TGFβRII, the only type II receptor for TGFβ ligands. Unexpectedly, mice that lack ALK5 in cartilage exhibit considerably more severe defects than do TGFβRII mutant mice (Michael O et al. Dev Biol. 2006 Aug. 15; 296(2):363-74) [6][5]. Consistent with a unique mode of action of ALK5 in cartilage, loss of ALK5 in articular cartilage causes severe OA in a genetic mouse model (Wang et al., Osteoarthritis Cartilage, 2017 November; 25(11):1868-1879) [6], However, cartilage specific deletion of TGFβRII prevents the development of OA (Chen et al., Am J Pathol. 2015 November; 185(11):2875-85) [7]. The findings that loss of ALK5 causes destruction of growth plate and articular cartilage while loss of TGFβRII is protective indicates that ALK5 may act through a unique and essential signaling pathway that is independent of TGFβRII, and therefore TGFβ signaling, to protect cartilage during development and in the progression of OA.

The type I BMP receptor ALK1 plays an essential role in angiogenesis (Oh et al., Proc. Natl. Acad. Sci. USA 2000, 97, 2626-2631; Umess et al., Nat. Genet. 2000, 26, 328-331) [8,9]. Loss-of-function mutations of ALK1 cause the vascular disease hereditary hemorrhagic telangiectasia (HHT, or Osler-Rendu-Weber syndrome) in humans. However, whether or not ALK1 had a role in cartilage and bone was not clear. As discussed above, it has been reported that a decreased ALK5/ALK1 ratio is correlated with OA [3]. This was tested by generating mice lacking ALK1 or both ALK1 and ALK5 in cartilage. It was reported that loss of ALK1 had no obvious impact on skeletal development in mice. However the severe cartilage defects seen in mice lacking ALK5 in cartilage are largely rescued in ALK1/ALK5 double mutants (Wang et al., PNAS 2019) [4]. Taken together, these findings provide evidence that the main role of ALK5 in cartilage is to inhibit ALK1 activation, and excessive ALK1 activity leads to cartilage destruction.

Ligands that bind to ALK1 at physiological concentrations are BMP9 and BMP10 (Chen et al, PNAS 2013) [10]. In vivo assays showed that ALK5 directly binds ALK1 and prevents ALK1 from complexing with the type II BMP receptor ACTRIIB in cartilage (Wang et al., PNAS 2019) [4]. ActRIIB has 300-fold higher affinity for BMP9 than does ActRIIA (Townson et. al., J Biol Chem. 2012 Aug. 10; 287(33):27313-2) [11]. The formation of ALK1-ActRIIB complexes is associated with the elevation of levels of pSmad1/5-mediated BMP signaling in cartilage (Wang et al., PNAS 2009)[4]. Consistently, in vitro luciferase reporter assays showed that BMP9 has higher activity in stimulating the BMP reporter in the chondrocyte cells that lack ALK5. The elevated BMP9 stimulation in Alk5-deficient cells is restored to normal by inactivating ALK1 in these cells, using an anti-ALK1 antibody treatment, or the BMP9 ligand trap protein ALK1-Fc. These results indicate that restricting the formation of ALK1-ActRIIB complexes which have high affinity with BMP9 can prevent excessive BMP signaling in cartilage. Furthermore, since loss of ALK5 causes OA and ALK5 antagonizes ALK1, these above results provide evidence that blocking ALK1 signaling may prevent OA development.

Therefore, we tested the hypothesis that blockade of BMP receptor ALK1 activity can prevent the development of post-traumatic OA (PTOA). In this disclosure we demonstrate that loss of the BMP type I receptor ALK1 in cartilage did not impact the structure or function of growth plate or articular cartilage; however, these mice are protected from developing the stigmata of OA when subjected to destabilization of the medial meniscus (DMM) surgery, a well-characterized model of PTOA. In this disclosure, we also demonstrate that injection of a BMP9/BMP10 ligand trap ALK1-Fc protein into the joint space of mice following DMM surgery prevents the development of PTOA.

Recombinant Human ALK-1 Fc Chimera Protein (R&Dsystems, Cat. 370-AL-100) acts as an inhibitor of ALK1. ALK1-Fc comprises a ligand-binding portion of the extracellular domain of ALK1 and a human IgG1. ALK1-Fc binds BMP9 and BMP10 with high affinity and blocks these ligands from interacting with receptors ALK1 and ALK2. A similar ALK1-Fc fusion protein (dalantercept) has been developed as a treatment for certain cancers, and is patented to inhibit angiogenesis and increase pericyte coverage in vascularized tissues, including tumors and the retina (Acceleron Pharma Inc, U.S. Pat. No. 8,158,584B2). Although not efficacious as a cancer therapy, ALK1-Fc has been shown to be safe and well tolerated in humans in phase II clinical trials. Considering the observed chondroprotective effects and known safety profile of this medication in humans, ALK1-Fc holds great promise as an intra-articular therapeutic agent for the treatment of OA.

Embodiments of the invention demonstrate the use of an in vitro system for evaluating the biological efficacy of ALK1 antagonists in chondrocytes with genetic defects mimicking OA conditions. As described herein, the ALK1 receptor normally binds to ALK5 and/or ActRIIA in healthy chondrocytes, but in OA conditions ALK5 expression levels decline in chondrocytes and ALK1 tends to complex with ActRIIB; this ALK1/ActRIIB complex has high affinity for BMP9 and BMP10. Because these ligands are present in the general circulation, the elevated level of ALK1/ActRIIB complex formation seen during the progression of OA leads to elevated BMP signaling and consequent cartilage destruction and heterotopic bone formation. Because it is difficult to test the optimal dose of ALK1 antagonists to block ALK1/ActRIIB complex formation and activity in vivo, an in vitro system is required. The invention provides solutions to this challenge and demonstrates that a genetic deletion of the ALK5 gene in chondrocytic cell lines, such as the ATDC5 mouse chondrocyte cell line can mimic the OA condition in which ALK1 complexes with ActRIIB and high BMP signaling output is activated by BMP9. The ALK5 gene is deleted using a CRISPR-Cas9 Gene Editing system, in which oligos that target ALK5 were synthesized and linked to lentiCRISPRv2 (1-vector system) plasmid, followed by transfection of this plasmid into the chondrocytic cells. BMP signaling output is monitored by a BMP signal luciferase reporter plasmid which contains a Smad1/5/8 response element in the promoter region. The biological efficacy of ALK1 antagonists is measured by their ability to inhibit BMP-9-induced BMP reporter activity by ALK5-deficient chondrocytic cells, such as ALK5-deficient ATDC5 cells. Embodiments of the invention include pharmaceutical preparations comprising the ALK1-Fc fusion protein wherein the median effective concentration (EC50) to block BMP activity in vitro is 45 ng/mL in the presence of 2 ng/mL of rhBMP-9; the EC50 for this effect of the ALK1 kinase inhibitor LDN-214117 is 40 nM in the presence of 2 ng/mL of rhBMP-9. Such pharmaceutically effective compositions may be formulated to be appropriate for administration to OA joints and tissues. The disclosed pharmaceutical effective doses may be used for protecting cartilage from degradation, inhibiting osteophyte formation, blocking abnormal ossification and heterotopic bone formation in mammals.

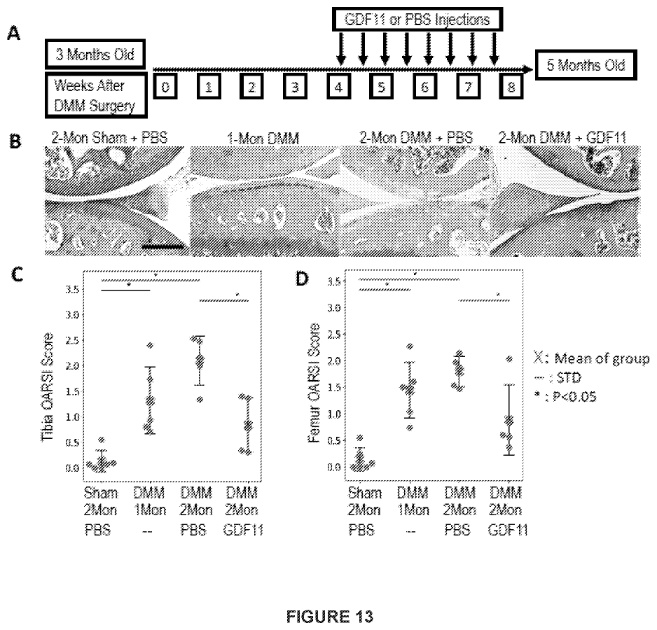

Growth differentiation factor 11 (“GDF11”, see, e.g., NCBI Reference Sequence: NP_005802.1) competes with BMP9/10 for binding to BMP type II receptors ACVRIIA or ACVRIIB, thereby preventing the association of BMP type I receptor ALK1 with ACVRIIA/B and decreasing BMP signaling. See, e.g., Moigneul et al., Nature Aging volume 3, pages 213-228 (2023). While the full length GDF protein is 407 amino acids, it is known in the art that GDF polypeptides/fragments also exhibit the functional activity discussed herein. The data presented in FIG. 13 shows that GDF11 polypeptides can attenuate the degeneration of articular cartilage in mice with established osteoarthritis, and that GDF11 can increase TGFbeta signaling. The GDF11 protein used in these experiments was obtained from Bio-techne/R&D, catalog #: 1958-GD, Asn299-Ser407, a peptide which has the same sequence one also commercially available from Peprotech (PMC10154197). These GDF polypeptides are truncated versions of the active protein having just 109 amino acids, and the ability to activate their receptors, similar to the full-length GDF11 protein (407 amino acids). In this context, as used herein, the term “GDF11 polypeptide” includes the full length protein and functionally active fragments/segments such as those used in the experiments that generated the data shown in FIG. 13.

Embodiments of the invention include methods of promoting cartilage preservation, repair, and/or regeneration in a mammal (typically a human diagnosed with osteoarthritis), the method comprising administering to the mammal a therapeutically effective amount of at least one agent selected from an ALK1-Fc fusion protein, a growth differentiation factor 11 (GDF11) polypeptide and LDN-214117, such that cartilage preservation, repair, and/or regeneration is promoted. In embodiments of the invention, the mammal can be administered an amount of agent(s) selected to be sufficient to inhibit heterotopic ossification, osteophyte formation, and/or destruction of articular cartilage in vivo. In certain embodiments, administered the agent intra-articularly.

Embodiments of the invention include methods of mitigating patient pain associated with damage to articular cartilage, the method comprising combining the articular cartilage with amounts of at least one agent selected from ALK1-Fc and LDN-214117 selected to be sufficient to inhibit pain in the patient following trauma to the articular cartilage; and inhibit degradation of the articular cartilage following trauma to the articular cartilage; or inhibit development and/or progression of osteoarthritis following trauma to the articular cartilage; such that pain associated with damage to articular cartilage is mitigated. Typically in such methods, the articular cartilage is combined with the agent by intra-articular injection of the agent. Typically the articular cartilage is disposed in a human diagnosed with osteoarthritis (OA). In certain embodiments, both ALK1-Fc and LDN-214117 are administered to the patient and/or ALK1-Fc is administered by intra-articular injection in amounts of 300 ng-500 ng per dose per week for at least 1-8 weeks. Related embodiments of the invention include methods of modulating the physiology of an articular cartilage, the method comprising combining the articular cartilage with amounts of at least one agent selected from ALK1-Fc, a growth differentiation factor 11 (GDF11) polypeptide and LDN-214117 selected to be sufficient to: inhibit fibrocartilage formation; inhibit osteophyte formation; thereby modulating the physiology of articular cartilage. Embodiments of the invention also include compositions of matter comprising at least one agent selected from ALK1-Fc, a growth differentiation factor 11 (GDF11) polypeptide and LDN-214117, wherein amounts of the agent in the compositions is sufficient to inhibit development and/or progression of osteoarthritis in a patient following trauma to the articular cartilage when the composition is disposed in the patient.

This disclosure further shows that an antibody detecting pSmad1/5 (Cell Signaling; 9516S) can be used for measuring the BMP signaling in articular cartilage. This shows that the pSmad1/5 level is increased in articular cartilage of mice lacking ALK5 in cartilage. In addition, the disclosure demonstrates that ALK1-Fc may be administrated by injecting into the cavity of the knee joint of mice, and the dose of 400 ng/per joint can effectively block pSmad1/5/8 elevation in DMM—induce OA articular cartilage and restores the BMP signal to normal levels.

The present invention also relates in certain embodiments to pharmaceutical compositions containing the selected agents that are disclosed herein. In one embodiment, the pharmaceutical composition comprises an ALK1-Fc fusion protein or GDF polypeptide or a small molecule such as LDN-214117 in a pharmaceutically acceptable excipient, carrier or diluent and in an amount effective to prevent or attenuate OA when administered to an animal, preferably a mammal, most preferably a human. In other embodiments, the pharmaceutical composition comprises an ALK1-Fc polypeptide in a pharmaceutically acceptable excipient, carrier or diluent and in an amount effective to treat a subject suffering from OA, for instance, in a method comprising administering to the subject an effective amount of an ALK1-Fc polypeptide disclosed herein.

There are a variety of different drug delivery (carrier) systems for osteoarthritis treatments can be adapted for use with embodiments of the invention including microparticles, nanoparticles, liposomes and hydrogels (see, e.g., Cao et al., Intra-Articular Drug Delivery for Osteoarthritis Treatment Pharmaceutics. 2021 December; 13(12): 2166, and Ma et al., Drug Des Devel Ther. 2022 May 4:16:1311-1347. Microparticles are micro-sized particles that can encapsulate drugs and release them slowly in the joint. Examples of microparticles include PLGA, PEA, PHBCL, gelatin, chitosan, heparin, and silver alginate microspheres. Nanoparticles are nano-sized particles that can penetrate biological barriers and improve the bioavailability of drugs. Examples of nanoparticles include polymeric nanoparticles, such as PAMAM dendrimers and poly(2-hydroxyethyl methacrylate)-pyridine nanoparticles, inorganic nanoparticles, such as gold, manganese dioxide, and mesoporous silica nanoparticles, and protein nanoparticles, such as lectin-cholesterol liposomes. Liposomes are spherical lipid bilayers that can encapsulate hydrophilic or hydrophobic drugs and provide supplementary boundary lubricants to the joint. Examples of liposomes include cationic liposomes, PEGylated liposomes, and PMPC-grafted liposomes. Hydrogels are three-dimensional and porous frameworks that can encapsulate and deliver drugs, proteins, and cells. Examples of hydrogels GelMA, chitosan, alginate, and PEG hydrogels.

Administration of the selected agent can be carried out via any of the accepted modes of administration of agents for serving similar utilities. The pharmaceutical composition can be prepared by combining an agent with an appropriate pharmaceutically acceptable carrier, diluent or excipient. Pharmaceutical compositions are formulated so as to allow the active ingredients contained therein to be bioavailable upon administration of the composition to a patient. The composition to be administered will, in any event, contain a therapeutically effective amount of an agent for treatment of a disease or condition of interest in accordance with the present teachings.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in Remington's Pharmaceutical Sciences (Mack Pub. Co., N.J. current edition).

The ALK1-Fc polypeptide is administered in a therapeutically effective amount, which will vary depending upon a variety of factors including the activity of the specific polypeptide; the metabolic stability and length of action of ALK1-Fc polypeptide; the age, body weight, general health, sex, and diet of the patient; the mode and time of administration; the rate of excretion; the drug combination; the severity of the particular disorder or condition; and the subject undergoing therapy. The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16^{th} edition, Merck and Co., Rahway, N.J., 1992; Goodman et al., eds., Goodman and Gilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)).

The total dose required for each treatment can be administered by multiple doses or in a single dose over the course of the day, if desired. Generally, treatment is initiated with smaller dosages, which are less than the optimum dose of the ALK1-Fc polypeptide. Thereafter, the dosage is increased by small increments until the optimum effect under the circumstances is reached. The ALK1-Fc polypeptide can be administered alone or in conjunction with other diagnostics and/or pharmaceuticals directed to the pathology, or directed to other symptoms of the pathology. The recipients of administration of the ALK1-Fc polypeptide can be any vertebrate animal, such as mammals.

The ALK1-Fc fusion proteins, GDF-11 polypeptides and small molecules can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al., Regional Anesthesia 22 (6): 543-551 (1997), all of which are incorporated herein by reference.

References listed above:
[1] www.cdc.gov/arthritis, n.d.
[2] C. M. Torio, B. J. Moore, National Inpatient Hospital Costs: The Most Expensive Conditions by Payer, 2013: Statistical Brief #204, in: Healthcare Cost and Utilization Project (HCUP) Statistical Briefs, Agency for Healthcare Research and Quality (US), Rockville (MD), 2006. www.ncbi.nlm.nih.gov/books/NBK368492/.
[3] E. N. Blaney Davidson, D. F. G. Remst, E. L. Vitters, H. M. van Beuningen, A. B. Blom, M.-J. Goumans, W. B. van den Berg, P. M. van der Kraan, Increase in ALK1/ALK5 Ratio as a Cause for Elevated MMP-13 Expression in Osteoarthritis in Humans and Mice, The Journal of Immunology. 182 (2009) 7937-7945.
[4] W. Wang, H. Chun, J. Baek, J. E. Sadik, A. Shirazyan, P. Razavi, N. Lopez, K. M. Lyons, The TGFβ type I receptor TGFβRI functions as an inhibitor of BMP signaling in cartilage, Proceedings of the National Academy of Sciences. 116 (2019) 15570-15579.
[5] M. O. Baffi, M. A. Moran, R. Serra, Tgfbr2 regulates the maintenance of boundaries in the axial skeleton, Developmental Biology. 296 (2006) 363-374.
[6] Q. Wang, Q. Y. Tan, W. Xu, H. B. Qi, D. Chen, S. Zhou, Z. H. Ni, L. Kuang, J. Y. Guo, J. L. Huang, X. X. Wang, Z. Q. Wang, N. Su, L. Chen, B. Chen, W. L. Jiang, Y. Gao, H. G. Chen, X. L. Du, Y. L. Xie, L. Chen, Cartilage-specific deletion of Alk5 gene results in a progressive osteoarthritis-like phenotype in mice, Osteoarthritis and Cartilage. 25 (2017) 1868-1879.
[7] R. Chen, M. Mian, M. Fu, J. Y. Zhao, L. Yang, Y. Li, L. Xu, Attenuation of the Progression of Articular Cartilage Degeneration by Inhibition of TGF-β1 Signaling in a Mouse Model of Osteoarthritis, The American Journal of Pathology. 185 (2015) 2875-2885.
[8] S. P. Oh, T. Seki, K. A. Goss, T. Imamura, Y. Yi, P. K. Donahoe, L. Li, K. Miyazono, P. ten Dijke, S. Kim, E. Li, Activin receptor-like kinase 1 modulates transforming growth factor-beta 1 signaling in the regulation of angiogenesis, Proceedings of the National Academy of Sciences. 97 (2000) 2626-2631.
[9] L. D. Urness, L. K. Sorensen, D. Y. Li, Arteriovenous malformations in mice lacking activin receptor-like kinase-1, Nature Genetics. 26 (2000) 328-331.
[10] H. Chen, J. Brady Ridgway, T. Sai, J. Lai, S. Warming, H. Chen, M. Roose-Girma, G. Zhang, W. Shou, M. Yan, Context-dependent signaling defines roles of BMP9 and BMP10 in embryonic and postnatal development, Proceedings of the National Academy of Sciences. 110 (2013) 11887-11892.
[11] S. A. Townson, E. Martinez-Hackert, C. Greppi, P. Lowden, D. Sako, J. Liu, J. A. Ucran, K. Liharska, K. W. Underwood, J. Seehra, R. Kumar, A. V. Grinberg, Specificity and Structure of a High Affinity Activin Receptor-like Kinase 1 (ALK1) Signaling Complex, Journal of Biological Chemistry. 287 (2012) 27313-27325.

### EXAMPLES

### Example 1: Targeting BMP Receptor ALK1 for the Prevention and Treatment of Post-Traumatic Osteoarthritis

Osteoarthritis (OA) is the most common degenerative joint condition worldwide affecting nearly 10% of the global population. The United States alone has over 32.5 million people suffering from OA and globally annual spending on the treatment of OA exceeds $185 billion (1). OA affects the entire joint, but degradation of articular cartilage (AC) is the primary factor impacting joint function (2, 3). Existing treatments for OA typically involve non-steroidal anti-inflammatory drugs (NSAIDs) including selective cyclooxygenase (COX)-2 inhibitors, which attempt to decrease inflammatory mediators associated with OA (4). Although many of these treatments are relatively successful in decreasing pain, none of these approaches restore native joint tissue architecture. Thus, currently, there are no disease-modifying treatments for OA (5).

The molecular pathways that regulate chondrogenesis pose an attractive therapeutic target for OA due to the central role of articular cartilage in OA progression. The degeneration of articular cartilage is often accompanied by the formation of heterotopic bone contiguous with the joint surface, known as osteophytes (6, 7). The transforming growth factor beta (TGFβ) and bone morphogenetic protein (BMP) signaling pathways are two of the most prominent ones in bone and cartilage, playing essential roles in the maintenance of these tissues (8). In a genome-wide meta-analysis across various osteoarthritis phenotypes in 826,690 individuals, multiple mutations in the TGFβ or BMP signaling pathway had an association with osteoarthritic joints (9). However, the roles of TGFβ signaling components have been difficult to decipher genetically owing to divergent effects on different joint tissues (10-12). TGFβ signaling is essential in mediating the formation of extracellular matrix components of cartilage, including type II collagen and aggrecan (13). Mice lacking the canonical TGFβ mediator Smad3^{-/-} globally or mice lacking Smad3 in cartilage (Smad3^{Col2}) each develop OA-like pathologies as adults (14, 15). The extent to which this reflects a role in adult articular cartilage homeostasis, or an earlier role in growth plate maintenance is unclear, but Smad3 represses MMP13 expression in articular cartilage, suggesting a direct protective function in this tissue (15). Even within cartilage, loss of TGFβ signaling components has distinct effects at different stages of development and OA progression. For example, loss of the type II TGFβ receptor TGFβRII in young (2 week old) mice leads to OA, but loss in mature articular cartilage is protective against OA (16, 17). In addition, loss of TGFβ type I receptor ALK5 in mature articular cartilage leads to severe OA, suggesting that ALK5 may have a role independent of TGFβRII in the maintenance of articular cartilage (18).

Complex effects have also been noted for bone morphogenetic protein (BMP) signaling pathways in articular cartilage. Analysis of loss of function phenotypes for BMP pathway components, such as type I BMP receptors (ALKs 1, 2, 6) or Smads1/5/8 in adult articular cartilage have not been performed, and direct roles in articular cartilage cannot be inferred from studies of gene ablation in cartilage tissues of embryos or immature animals, owing to widespread effects on growth plate cartilage (19-22). Nonetheless, numerous studies point to a role for BMP signaling in OA progression (23, 24). Some BMP ligands, such as BMP2, promote expression of markers of chondrocyte hypertrophy and catabolic genes such as MMP13 in AC(25). However, whether a specific BMP triggers terminal differentiation or maintains AC is dependent on the target cells, timing, and mode of delivery (26, 27). Several studies have explored antagonism of BMP signaling as a therapy for OA (24, 25, 28, 29). These studies show that antagonism of BMP receptor activity may be a promising therapeutic option. For example, the small molecule kinase inhibitor LDN-193189, which targets the type I BMP receptors ALK2 and ALK3, prevented chondrocyte hypertrophy and MMP13 expression ex vivo and in vivo (24, 29, 30). On the other hand, LDN-193189 was not able to prevent chondrocyte hypertrophy in bone marrow stromal cell-derived chondrocytes (31). Taken together, these findings support the possibility of BMP receptor antagonism as a treatment for OA, but also highlight the need for additional in vivo studies.

ALK1 is a target of particular interest because there is evidence that TGFβ/BMP crosstalk mediated by ALK1 and the TGFβ receptor ALK5 regulates OA progression. The ALK1/ALK5 expression ratio is elevated in OA cartilage compared to healthy articular cartilage, accompanied by an increase in BMP signaling in AC (25, 32-34). ALK1 has been correlated with hypertrophy and the production of MMP13 in vitro (32). We showed previously that ALK1 and ALK5 exert antagonistic functions in growth plate cartilage; loss of ALK5 in cartilage led to severe chondrodysplasia, which was largely corrected in ALK1/ALK5 double mutants (35). However, cartilage-specific loss of ALK1 alone had no obvious consequences (35). These findings provide evidence that ALK1 is not required for chondrogenesis but can drive pathological BMP signaling if unregulated. Mechanistically, we found that in growth plate cartilage, ALK5 sequesters the type II BMP receptor ActRIIB into ALK5/ActRIIB complexes, preventing the formation of ALK1/ActRIIB complexes (35). Although most type I BMP receptors bind multiple ligands, the circulating ligands BMPs 9 and 10 are the only ones that bind ALK1 at physiological concentrations, and of the potential receptor complexes for BMP9, ALK1/ActRIIB has the highest affinity for BMP 9 (36). In summary, among the BMP receptors, ALK1 is a unique target because it is the only type I BMP receptor whose loss in growth plate cartilage doesn't cause obvious pathologies (35), its expression is highly associated with OA cartilage (32), and it plays a significant role in crosstalk between TGFβ and BMP signaling in cartilage tissues (25).

Based on these findings, we hypothesized that inhibition of BMP signaling through ALK1 might protect against OA progression. However, the in vivo role of ALK1 in the maintenance of mature cartilage is unknown. We address this question through the deletion of ALK1 in cartilage using Col2a1-Cre; Alk1^{f/f} mice. In addition, we applied pharmaceutical agents that inhibit ALK1 activation to mice after undergoing DMM surgery. LDN-214117 is a potent small molecule kinase inhibitor that preferentially inhibits ALK1 and ALK2 (IC₅₀=24 nM) over the BMP receptor ALK3 (IC₅₀=1,171 nM) (37, 38). LDN-214117 is well tolerated and patented for treatment of fibrodysplasia ossificans progressive (39). The ligand trap ALK1-Fc consists of the extracellular ligand binding domain of ALK1 fused to an immunoglobulin Fc domain. It potently and selectively binds BMPs 9 and 10, which only act through ALK1 and ALK2; ALK1-Fc shows no affinity for other BMPs (40-43). ALK1-Fc is a large molecule that contains a human IgG1 domain and is therefore unable to easily diffuse into cartilage, which is distinct from small molecule inhibitors such as LDN-214117 (44, 45). Moreover, ALK1 ligands BMPs 9 and 10 are found in the systemic circulation but are not produced in cartilage, which potentially allows ALK1-Fc to prevent BMPs 9 and 10 from entering cartilage tissues (46). Although a Phase 2 clinical trial of ALK1-Fc (Dalantercept) did not demonstrate efficacy against renal cancer, it showed that systemically delivered ALK1-Fc is well tolerated in humans (47). We therefore investigated the role of ALK1 through genetic analysis and evaluated the impact of local administration of specialized ALK1/ALK2 inhibitors on OA progression.

### Results

### ALK1 is not Required for Articular Cartilage Maintenance, and Mediates AC Degeneration in Post-Traumatic OA

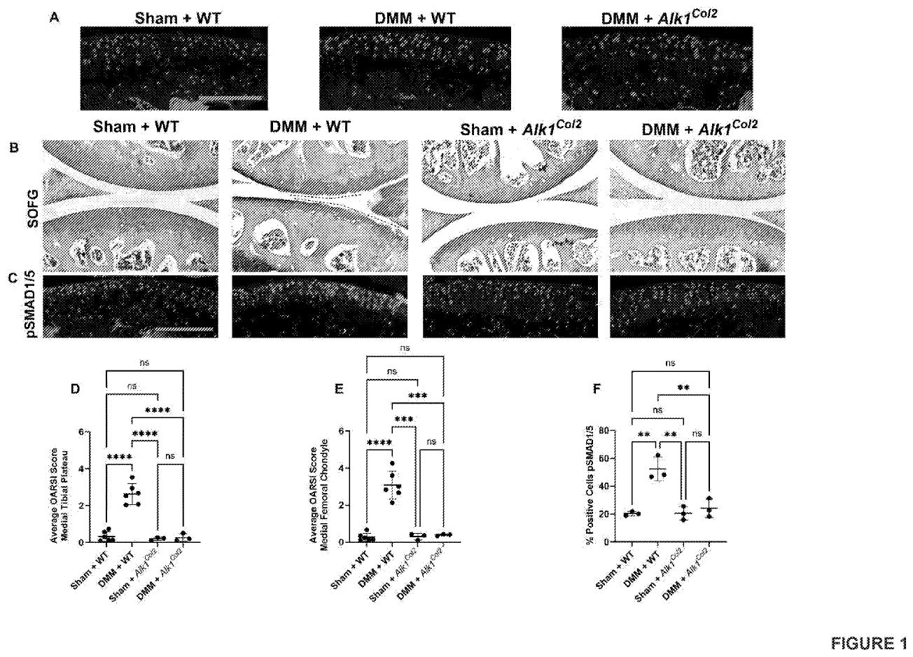

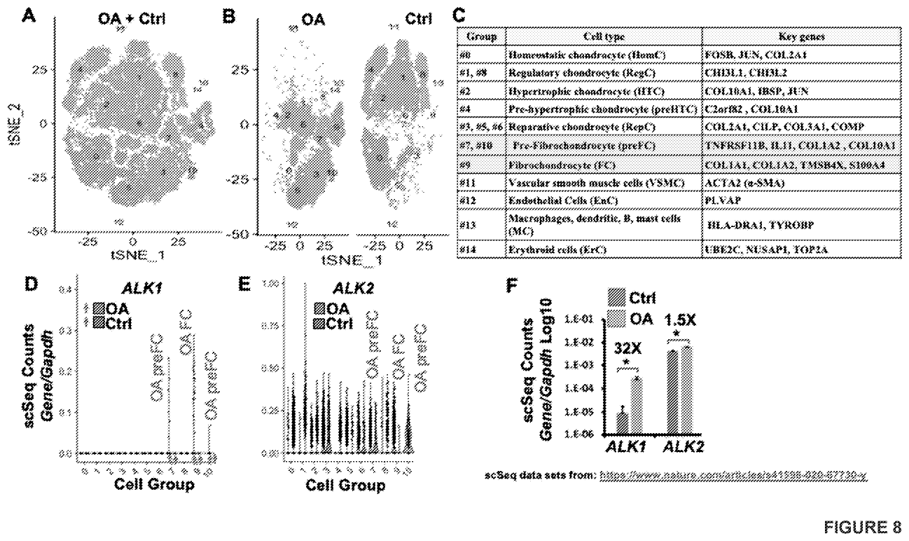

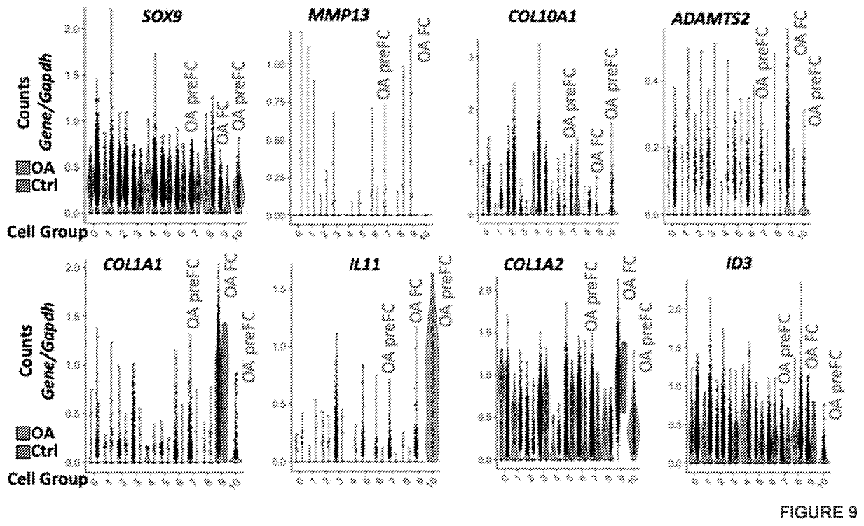

We employed the DMM-model of post-traumatic OA to evaluate the effect of ALK1 in the maintenance of articular cartilage after trauma, where the medial meniscotibial ligament is transected in mice to produce an osteoarthritic phenotype (48). As an initial approach, expression of ALK1 was assessed by immunofluorescence (IF) in healthy articular cartilage of wild-type mice that received sham surgery, and in OA cartilage of mice receiving DMM surgery. At the protein level, ALK1 was undetectable by IF in healthy articular cartilage but was present in a subset of cells in the superficial and middle zones of OA cartilage (FIG. 1A). We also examined a scRNASeq database derived from human OA chondrocytes for Alk1 expression. Alk1 mRNA expression was enriched in OA pre-fibrochondrocytes and fibrochondrocytes (FIG. 8A-D), and the expression level increased 32-fold in OA cartilage compared to intact control cartilage (FIG. 8F) (49). Although Alk2 was expressed in all OA and control cell populations, there was a 1.5× increase in Alk2 levels in OA chondrocytes compared to control chondrocytes (FIGS. 8E,F). Furthermore, cluster analysis revealed that pre-fibrochondrocytes and fibrochondrocytes in OA cartilage are SOX9-positive cells (FIG. 9) that specifically expressed Ak1 (FIG. 8D). These cells in OA cartilage were enriched in the expression of terminally differentiated markers (MMP13, COL10A1, ADAMTS2) (50), as well as fibrotic markers (COL1A1, IL11, COL1A2) (FIG. 9) (7, 49). This observation provides evidences that Alk1 expression is associated with increased expression of OA markers. Additionally, Alk1-positive cells exhibited elevated expression of ID3 in OA compared to control cells (FIG. 9). ID3 is a downstream target of BMP signaling, consistent with the hypothesis that Alk1-positive cells exhibit increased BMP activity.

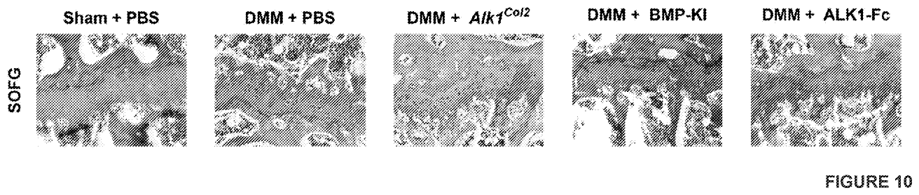

To test the potential function of ALK1 in articular cartilage, we generated mice lacking ALK1 using Col2a1-Cre (51). As reported previously, Alk1^{Col2} mice were viable and exhibited no obvious defects in the growth plate (35) (FIG. 10) and in the articular cartilage (FIG. 1). We next performed DMM surgery on 12-week-old male mice and examined the structure of the AC at 5 months (20 weeks) of age. DMM surgery led to a loss of surface cartilage and AC fissuring in control mice compared to sham-operated controls (FIG. 1B; compare WT/Sham and WT/DMM). However, Alk1^{Col2} mice exhibited little cartilage erosion or proteoglycan loss following DMM surgery (FIG. 1B; compare WT/DMM and Alk1^{Col2}/DMM) Osteoarthritis Research Society International (OARSI) histopathology scoring of the medial tibial plateau and medial femoral condyle confirmed that Alk1^{Col2} mice were protected against cartilage degradation following DMM surgery, as there was no significant difference between the OARSI scores of Alk1^{Col2} mice that received sham or DMM surgery (FIG. 1D-E). Expression of the canonical BMP pathway mediators pSMAD1/5 confirmed that DMM surgery led to elevated BMP signaling in WT mice but not in Alk1^{Col2} mice (FIG. 1C, F). Our genetic findings thus indicate that loss of ALK1 in AC is sufficient to reduce canonical BMP signaling and confer significant protection against OA progression.

Inhibition of ALK1/ALK2 Activity Prevents Articular Cartilage Degeneration after DMM Surgery

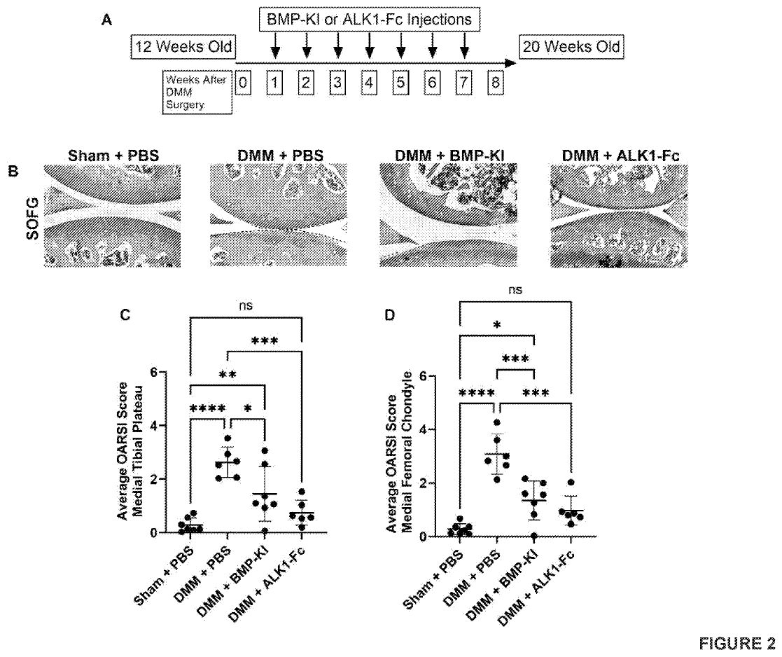

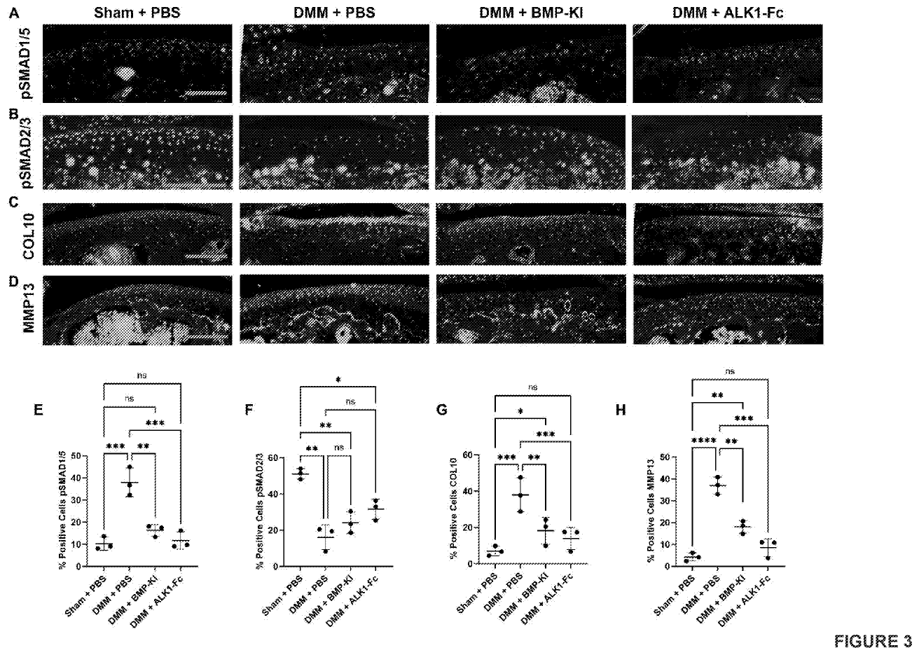

Our preliminary finding that Alk1^{Col2} mice are protected from DMM-induced cartilage damage but exhibit no obvious alterations in joint tissues under normal conditions suggested that pharmacological blockade of ALK1 activity may be an effective strategy against OA progression. We therefore injected either a BMP kinase inhibitor LDN-214117 (BMP-KI) or an ALK1 ligand trap (ALK1-Fc) into the knee joint and periarticular tissue in 12-week-old male mice (FIG. 2A) with a dosage based on the in vitro IC50 value provided by the manufacturer. As discussed previously, LDN-214117 is highly selective for ALK1 and ALK2 (biochemical IC50=24 nM) over ALK3 (biochemical IC50=1,171 nM) (37). We injected LDN-214117 (a total of 4.2 ng at a concentration of 200 nM, which has limited effect (<25% IC50) on ALK3) into the knee joint and periarticular tissues at the time of DMM surgery and weekly thereafter for an additional 7 weeks. Knee tissues were collected 8 weeks after surgery (FIG. 2A). ALK1-Fc specifically binds to and inhibits BMPs 9 and 10. These BMPs activate ALK1 and ALK2 but no other BMP/TGFβ receptors; moreover, BMPs 9 and 10 are the only ligands that activate ALK1 (52). Therefore, ALK1-Fc is expected to block all activation of ALK1 as well as BMP9/10-mediated activation of the structurally related receptor ALK2. ALK1-Fc (400 ng/injection; 16 ng/kg body weight) was administered using the same injection technique on the same schedule as LDN-214117 (BMP-KI) (FIG. 2A). Again, knee tissues were collected 8 weeks after surgery. Safranin-O/Fast-Green staining and OARSI scoring demonstrated protection of articular cartilage after inhibition of BMP signaling with either BMP-KI or ALK1-Fc treatment compared to PBS injection (FIG. 2B). OARSI scoring of the medial tibial plateau and medial femoral condyle confirmed this protection (FIG. 2C-D). As discussed previously, expression of the canonical BMP mediators pSMAD1/5 was elevated in WT mice but not in Alk1^{Col2} mice following DMM surgery (FIG. 1). Consistent with this finding, pSMAD1/5 expression was reduced to normal levels in WT mice treatment with BMP-KI or ALK1-Fc (FIG. 3A, E); however, expression of the canonical TGFβ pathway mediators pSMAD2/3 staining was not restored to normal levels (FIG. 3B,F). MMP13 and COL10 levels were visibly reduced in mice with BMP-KI or ALK1-Fc (FIG. 3C,D,G,H). Therefore, treatment with ALK1/2 inhibitors is associated with reduced BMP canonical pathway activity and with deceased expression of markers of hypertrophy and terminal differentiation but does not restore TGFβ pathway activity.

### Inhibition of ALK1/ALK2 Activity Following the Onset of OA Attenuates Articular Cartilage Degeneration

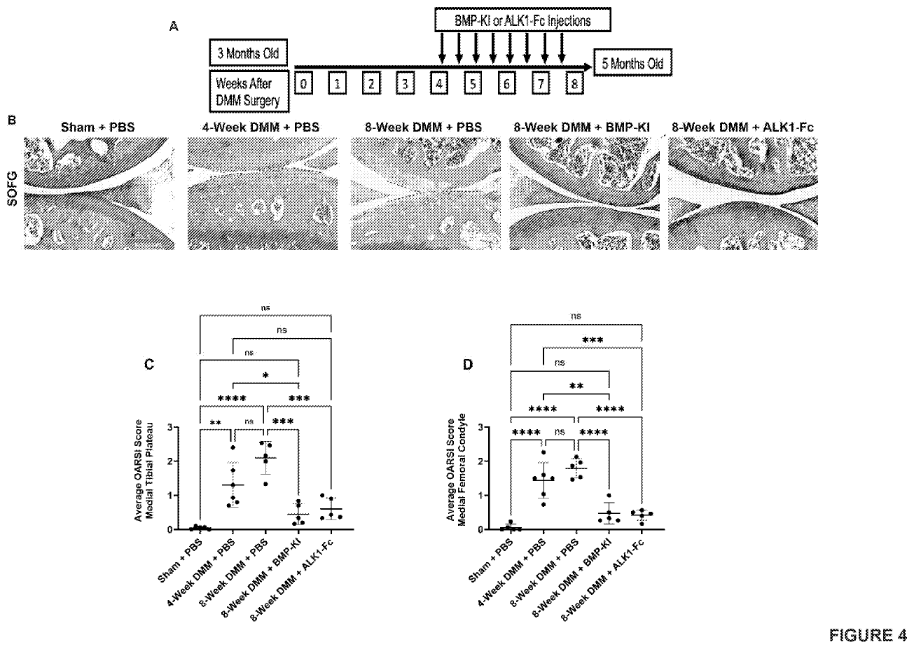

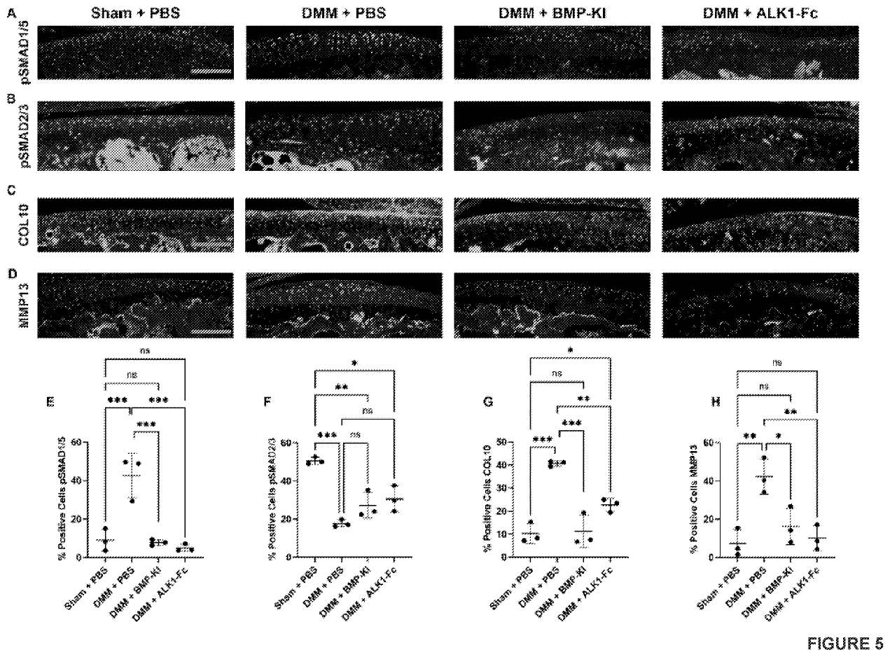

The above data indicate that blockade of BMP9/10-mediated ALK1/ALK2 activity beginning at the time of DMM surgery can prevent the onset of OA. To evaluate the effectiveness of blocking ALK1/ALK2 BMP signaling activity on the progression of established OA, we delayed administration of BMP-KI and ALK1-Fc until 4 weeks following DMM surgery (FIG. 4A). By 4 weeks post DMM, proteoglycan loss and articular surface fissures are evident in DMM mice treated with vehicle, particularly on the tibial plateau (FIG. 4B). OARSI scoring revealed a trend toward increased damage at 8 weeks compared to 4 weeks post DMM, although the difference was not significant (FIG. 4C,D). However, mice treated with BMP-KI or ALK1-Fc beginning 4 weeks after DMM surgery and examined 8 weeks after surgery exhibited significantly less damage than vehicle-treated mice examined 8 weeks after surgery (FIG. 4C, D), demonstrating a therapeutic effect. Interestingly, mice treated with BMP-KI appear to exhibit less damage on both the femoral and tibial surface than vehicle-treated mice examined 4 weeks after surgery (FIG. 4C, D). Mice treated with ALK1-Fc also exhibited less damage on the femoral surface compared to 4-week old mice, and a trend toward less damage on the tibial surface was observed (FIG. 4C, D). Overall, based on comparison of histology and OARSI scores at 4 weeks post DMM surgery, these findings provide evidence of a potential regenerative effect for BMP-KI, and possibly for ALK1-Fc following 4 weeks of inhibitor treatment. Immunofluorescent analysis indicated an attenuation of DMM-induced pSMAD1/5 signaling, but no significant rescue of decreased pSMAD2/3 signaling (FIG. 5A, B, E, F) following delayed treatment. A decrease in COL10 and MMP13 was also observed through immunofluorescence (FIG. 5C, D, G, H). Overall, these findings indicate that BMP-KI and ALK1-Fc can stop the progression of established OA.

### Inhibition of ALK1 Activity Reduces Osteophyte Formation

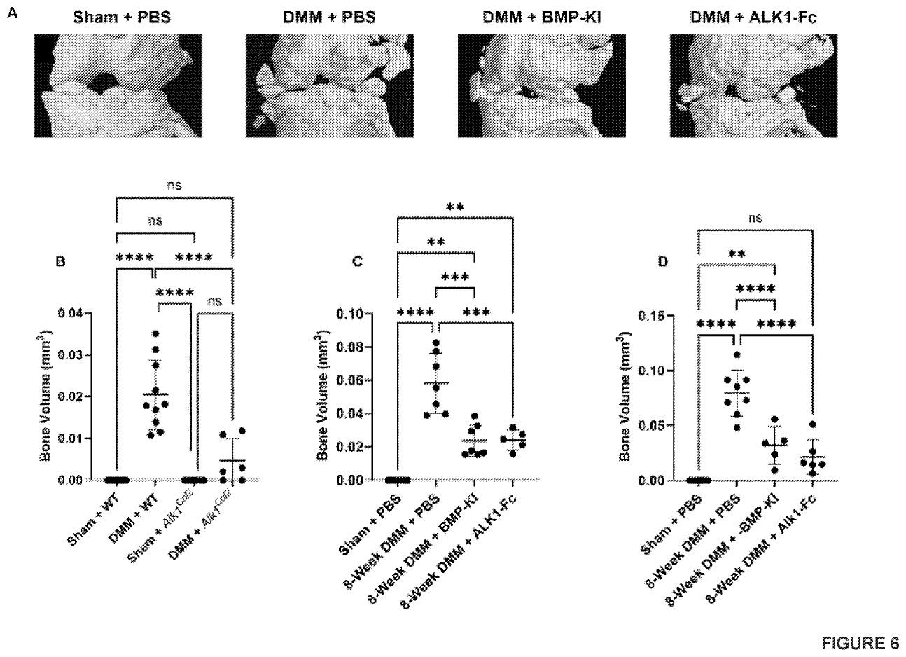

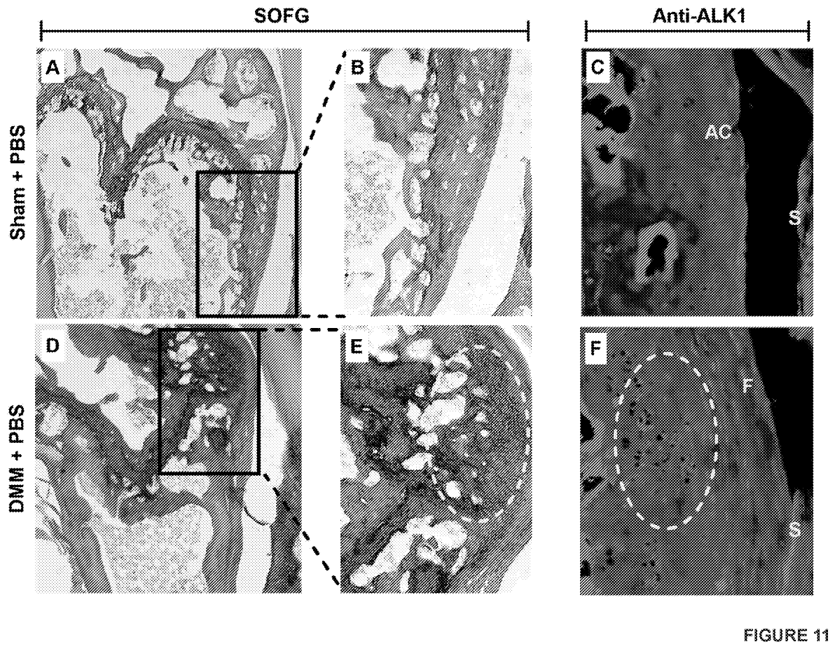

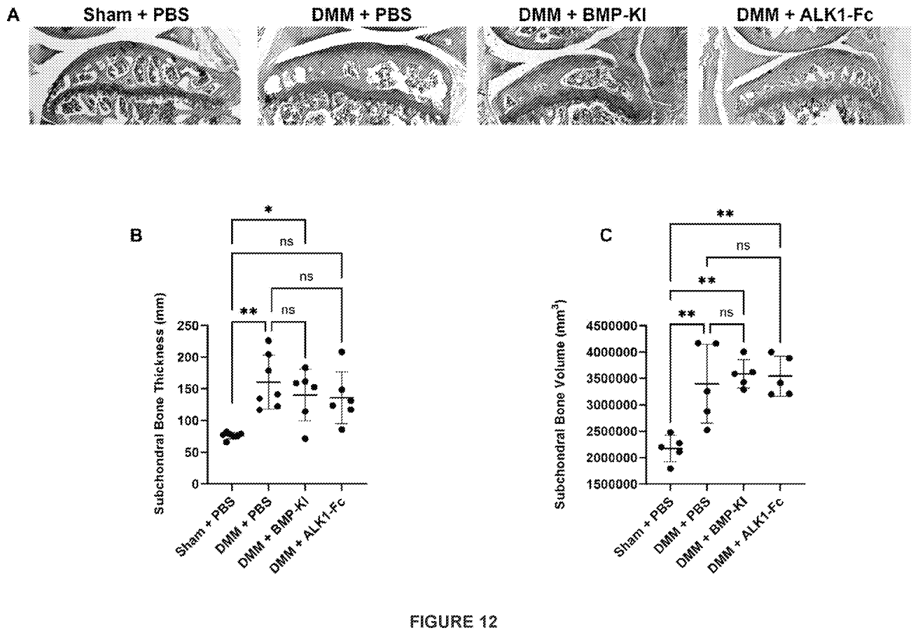

Osteophytes are fibrocartilage-capped bony outgrowths that typically arise at the margins of the joint surface at synovium-articular cartilage junctional zones. They are thought to develop in part from cells found in the periosteum (7, 53). BMP and TGFβ signaling pathways are strongly implicated in osteophyte formation and growth (6). Immunohistochemistry analysis revealed that ALK1 protein is enriched in osteophytic cartilage in DMM mice (FIG. 11). MicroCT analysis showed reduced osteophyte volume in Alk1^{Col2} DMM mice (FIG. 6B). In addition, MicroCT analysis demonstrated reduced osteophyte volume in DMM mice treated immediately after surgery with BMP-KI or ALK1-Fc when compared to the vehicle condition (FIG. 6A, C). When applied after a 1-month delay, BMP-KI and ALK1-Fc treated mice also exhibited a significant reduction in osteophyte volume (FIG. 6D). However, there was no observed effect of BMP-KI nor ALK1-Fc on the thickening of the subchondral bone plate after DMM surgery (FIG. 12).

### Inhibition of ALK1 Activity Reduces OA Associated Pain

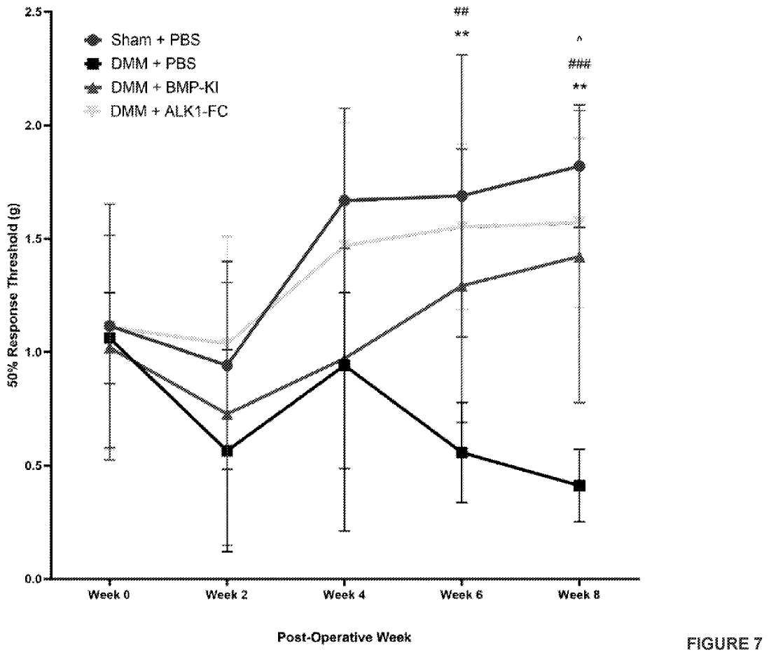

In a functional assessment of BMP-KI and ALK1-Fc, we measured pain and sensitivity in mice following DMM surgery and treatment with BMP-KI and ALK1-Fc using Von Frey analysis. Mice underwent DMM surgery at 3 months of age and were treated with BMP-KI or ALK1Fc once a week for 8 weeks following surgery. Mice underwent Von Frey testing immediately prior to surgery, and each week following surgery. Von Frey testing involved exposure of the injured limb hindfoot plantar surface to a monofilament of a designated caliber (measured in grams). Testing was based on the 50% paw withdrawal threshold (50% PWT), where exhibition of pain symptoms was observed in a minimum 5 out of 10 total exposures to the specified caliber of Von Frey monofilament. The 50% PWT was then calculated mathematically as previously described(54). A higher 50% paw withdrawal threshold indicates less sensitivity in the surgically treated limb. Mice undergoing sham surgery were also tested as a control. By week 6 after surgery, there was a significant decrease in pain in the sham group compared to the DMM control group, indicating that the pain associated with surgical incision had diminished, and the OA-associated pain could be distinguished between sham and DMM groups (FIG. 7). By week 6 after surgery, there was a significant decrease in pain in DMM mice treated with ALK1-Fc, and by week 8 after surgery, there was a significant decrease in pain in DMM mice treated with BMP-KI.

### Discussion

Osteoarthritis (OA) is the most common joint disease in the US (55). Symptomatic OA is associated with significant clinical morbidity, and, unfortunately, there are currently no FDA-approved disease modifying therapies. As a result, OA remains a significant unmet clinical need. The hallmark of OA is the breakdown of articular cartilage (55). Current treatments for OA only provide short-term relief and are primarily focused on mitigating the pain symptoms of OA, yet have no impact on the restoration of native joint tissue architecture (56). Moreover, the current non-surgical treatments for OA have been associated with a number of potential adverse effects (57), and surgical treatments are associated with perioperative pain, prolonged convalescence, and the potential for a variety of post-operative complications. Due to the inability of current non-surgical treatments to longitudinally and meaningfully reduce the disability associated with OA, therapeutic strategies that seek to protect and restore healthy joint microarchitecture are critical. In this study, we demonstrate that BMP9/10 signaling through the BMP receptor ALK1 is a key player in OA progression, and that inhibition of this pathway protects cartilage from further degradation after the induction of post-traumatic OA.

While TGFβ and BMP signaling pathways have been implicated in OA progression, their specific functions in OA cartilage in vivo remain unclear. Studies have outlined both the importance and complexity of TGFβ and BMP signaling pathways in the development of joint tissues, and have implicated perturbations in these signaling pathways as causing disease phenotypes within the joint (12, 34, 35, 58-60). Our rationale for examining the function of ALK1 in AC was the finding that the ALK1:ALK5 balance is shifted towards increased ALK1 mediated BMP signaling in OA cartilage (25, 32-34). Furthermore, we showed previously that loss of ALK1 in growth plate cartilage had no apparent deleterious effects (35). In contrast, loss of ALK5 in AC and/or growth plate cartilage is associated with severe cartilage degeneration (18, 35, 61). However, when both ALK1 and ALK5 were ablated in cartilage, many of the adverse effects seen in the ALK5 mutants were attenuated, supporting the hypothesis that a disturbance in the balance of ALK1:ALK5 signaling is a primary signaling mechanism that drives cartilage degradation (35). In our current study, a cartilage-specific knockout of ALK1 was protective against AC degradation using a surgical model of post-traumatic OA (DMM) in mice, supporting the concept that blocking ALK1 signaling has therapeutic potential for the prevention of OA. Two potential therapeutic strategies aimed at modulating BMP signaling were investigated: i) the ALK1 and ALK2 kinase inhibitor LDN-214117 (BMP-KI), and ii) the highly specific ligand trap, ALK1-Fc. Of note, ALK1-Fc has already been shown to be safe in phase 2 human clinical trials when administered systemically at doses much higher than the one we employed locally in this study (62).

A recent in vivo study by Jaswal and colleagues demonstrated that the BMP kinase inhibitor LDN-193189 was protective for AC cartilage in a post-traumatic OA model(24). LDN-193189 has high potency for inhibiting ALK1 and ALK2 (with IC50 values 6× and 20× lower) compared to ALK3 and ALK6, according to its IC50 values from kinase assays (0.8, 0.8, 5.3, 16.7 nM for ALK1, ALK2, ALK3, ALK6 respectively) (63). It is therefore possible at the doses administered that the therapeutic effect of LDN-193189 in OA observed in their study (24) is derived from the inhibition of ALK1 and ALK2, or from a combination of ALK1/2/3 (24). In comparison, LDN-214117, used in the current study, exhibits higher selectivity for ALK1 and ALK2 (with an IC50 48× lower compared to ALK3), and ALK1-Fc specifically inhibits BMPs 9/10, which exclusively act through ALK1 and ALK2.

Several previous studies reached contradictory conclusions regarding the role of ALK3 and ALK6 in adult AC, and their potential role in OA (64). For example, the downregulation of ALK3 and ALK6 has been associated with the degeneration of cartilage (65), and deficiency of ALK3 has been associated with the thinning of AC (bone-on-bone contact) at the joint surface (66). Furthermore, a correlation between the elevation of ALK3 and the worsening of cartilage defects has been noted (67). While LDN-193189 demonstrated no reduction of chondrogenic hypertrophic markers in vitro (31, 68), Jaswal et al. demonstrated a protective effect in vivo (24). Given the results of the current study, it is likely that the protective effects of LDN-193189 were due at least in part to inhibition of ALK1 and ALK2 (24, 29). Thus, our results, along with those of Jaswal et al (24) point to a role for ALK1 in OA progression, while also supporting a role for ALK2.

Given the pleiotropic effects of BMP signaling in joint tissues, therapeutic application of BMP inhibitors will require tissue specificity to achieve the intended effect and to minimize the potential for pathologic off target effects. ALK1 is an attractive target because it is elevated in OA cartilage, yet its loss in joint tissues derived from Col2a1-Cre-expressing cells (growth plate and articular cartilage, synovium, ligament, joint capsule) has no obvious impact on joint structure or function (FIG. 1B, FIG. 10) (35). The finding that ALK1 contributes to OA progression also implicates the circulating ligands BMP9 and BMP10, as these are the only ligands for ALK1 present in physiological concentrations, as well as the only ones that bind to ALK1-Fc(36, 70). Neither of these ligands has been shown to be produced in articular cartilage, but BMP9 is highly expressed in cholinergic nerves in the brain, and cholinergic nerves innervate joints affected by OA (70, 71). Furthermore, BMP9 has been detected in human and rat synovial tissue of knee joints (72), and in human synovial tissue of the temporomandibular join t(73). Whether BMP9 is present in synovial fluid, and whether cholinergic nerves innervating osteoarthritic joints express BMP9, warrants future investigation.

Injection of ALK1-Fc or LDN-214117 intra-articularly and peri-articularly at the knee joint each week for two months immediately after DMM surgery was sufficient to prevent degradation of AC compared to the vehicle condition. In order to more closely simulate the clinical presentation of OA, we also tested these therapeutics in DMM mice that already exhibited moderate OA. In this setting, ALK1-Fc and BMP-KI were both able to protect cartilage from further degradation. Interestingly, when compared to the DMM+PBS condition harvested 4 weeks after surgery (the timepoint at which treatment with BMP-KI and ALK1-Fc was initiated), there was a significant decrease in average OARSI scores in the medial femoral condyle for both therapeutics, and a decrease in the average OARSI score in the medial tibial plateau for treatment with BMP-KI, raising the possibility of a possible regenerative effect on the AC. Furthermore, pSMAD1/5 levels that had increased in the DMM condition were successfully attenuated after treatment with ALK1-Fc and BMP-KI. Protein markers of cartilage hypertrophy and degradation, such as MMP13 and COL10 were also significantly decreased compared to the DMM vehicle condition. However, we noted that pSMAD2/3 levels were not restored, and subchondral bone changes were not reversed, indicating that pharmacological blockade of BMP9/10 signaling alone may not be sufficient to address these pathologic processes. Long term studies would be needed to determine if these parameters can be corrected after a more prolonged exposure to these investigational compounds.

In addition to AC degeneration, there are other stigmata that are associated with the structural pathology and pain symptoms associated with OA. One of these is the formation of osteophytes. Osteophytes are fibrocartilage-capped bony outgrowths that arise from cells in the periosteum and synovium (7). As noted previously, ALK1 is enriched in fibrocartilage (FIG. 8) and in developing osteophytes (FIG. 11). Defects in cartilage have been associated with increased pain, and with pain progression, potentially due to loss of the cushioning effect between areas of subchondral bone, a highly innervated tissue type (74, 75). Osteophytes pose a unique challenge as they have been observed to contain a significant degree of sensory innervation (76, 77). We found a significant decrease in osteophytes volume after the application of BMP-KI and ALK1-Fc. Mechanistically inferences can be made about the role of ALK1 in osteophyte formation. As noted previously noted, ALK1 is enriched in fibrocartilage, a cell type that gives rise to osteophytes (also referred to as osteochondrophytes, related to their cartilage component) (6, 7), and BMP9 is one of the most osteoinductive members of the BMP family (78).

A functional pain analysis was conducted to supplement the histological observations of improvement to joint architecture. All experimental groups saw a significant increase in pain monitored by Von Frey analysis immediately after surgery (week 2), as expected, and progressively saw a decrease in pain between week 2 and weeks 4. By week 6, there was an increase in pain in the DMM control group, while the mice that received ALK1-Fc or BMP-KI did not exhibit an increase in pain. Furthermore, at week 8 there was no significant difference between ALK1-Fc or BMP-KI mice compared to mice that had received sham surgery. It is possible that this decrease in pain was facilitated by either preserving the AC layer which confers an improved ability to absorb loads between the tibia and femur, by protecting other joint tissues from the pathophysiologic mechanisms of OA, or a combination of these factors. It is also possible that ALK1 mediates OA pain through effects on vascular ingrowth and neuroinflammation in vascularized and innervated structures such as the bone, synovium or periosteum, as ALK1 is required for angiogenesis (46, 79).

This study has a number of limitations, including those inherent to the use of a surgically induced OA rodent model which may not fully recapitulate the pathobiology occurring in human OA. Furthermore, this study used a limited treatment duration (4 to 8 weeks), and thus the efficacy of longer-term delivery of these compounds is unknown. It is also worth noting that OA is a whole-joint disease. While ALK1-Fc and LDN-214117 protected AC and potentially facilitated some degree of cartilage tissue regeneration, neither inhibitor restored subchondral bone. During OA, the subchondral bone plate increases in thickness, likely due to persistent increased mechanical stress and the resultant changes in bone remodeling(80). While neither BMP-KI nor ALK1-Fc prevented subchondral bone sclerosis during the period under observation, neither therapeutic exacerbated it. Whether the subchondral bone can be restored after prolonged exposure to these inhibitors remains unknown. Other tissue changes in the joint, such as synovial inflammation, vascularization and nerve ingrowth remain to be characterized (81). Given the potential efficacy of ALK1-Fc and LDN-214117 demonstrated here, these additional studies are warranted. Moreover, the pharmacokinetic and pharmacodynamic properties associated with intra-articular administration of ALK1-Fc and LDN-214117 enabling optimization of the dose and timing of delivery need to be determined.

Multiple aspects of BMP signaling have yet to be adequately characterized in OA. While our analysis of Alk1^{Col2} mice shows unequivocally that ALK1 mediates damage to AC and osteophyte formation during OA, ALK1-Fc and LDN-214117 would also be expected to impact signaling through the structurally related ALK2 receptor. Therefore, additional genetic studies are needed to establish the extent to which ALK1-Fc and LDN-214117 mediate their effects through inhibition of ALK1 and/or ALK2. Finally, the mechanisms regulating the interrelationship between TGFβ and BMP signaling required to maintain AC health remain unclear. ALK1-Fc and BMP-KI, at the given doses and treatment duration, demonstrate a trend of elevating pSmad2/3 levels in the DMM articular cartilage but do not reach statistical significance compare to DMM control (FIGS. 3F and 5F). Re-establishment of the BMP/TGFβ signaling in AC may require coordinated inhibition of BMP signaling along with activation of TGFβ signaling in AC (82). The importance of maintaining appropriate levels of both TGFβ and BMP signaling in multiple joint tissues is further supported by previous studies showing that inhibiting TGFβ signaling in subchondral bone using an ALK5 inhibitor attenuated AC damage (83).

In summary, we have shown that ALK1 inhibition via LDN-214117 or ALK1-Fc are both effective strategies for the prevention of OA progression. Further studies are needed to investigate how these agents can be used in combination with modulators of TGFβ pathways to achieve a balanced BMP/TGFβ signaling.

### Methods

### DMM Surgeries.

Mice (C57BL/6J) were purchased from The Jackson Laboratory. All mice were housed on a 12-hour light/dark cycle with unrestricted access to standard mouse food and water. The DMM model of injury induced OA was performed on mice at 12 weeks of age by opening the joint capsule and transecting the medial meniscotibial ligament in the right knee, destabilizing the medial meniscus(48). A sham operation was performed on the left knee of each mouse by observing the medial meniscotibial ligament but not transecting it. Mice were administered carprofen 5 mg/kg (Zoetis; 1041283) subcutaneously 30 minutes prior to surgery, immediately post-surgery, and 24 hours post-surgery to minimize pain.

ALK1/2 kinase inhibitor LDN-214117, 4.2 ng (Sigma-Aldrich; SML1119-25MG), ALK1-Fc, 400 ng (R&D; 370-AL), or Phosphate Buffered Saline was administered via intraarticular and periarticular injection. Treatments were initiated either 1 day after DMM surgery once a week for 8 weeks, or 1 month after DMM surgery twice a week for 4 weeks. Mice were sacrificed at 20 weeks of age (2 months after surgery). To understand the level of articular cartilage damage 1 month after DMM surgery, a cohort of mice was sacrificed at 16 weeks old (FIG. 4).

### Histology

Knee joint samples were harvested and fixed in 4% Paraformaldehyde for 3 days, and subsequently decalcified in formic acid (Statlab; 1414-32) for 3 days and embedded in paraffin. Sagittal sections 7 micrometers in thickness were taken and stained using Safranin-O (Sigma-Aldrich; S8884-25G), fast green (Sigma-Aldrich; F7258-25G), and hematoxylin (Fisherbrand; 245-656) using a standard histology protocol. Images were taken with the Olympus BX60 brightfield microscope using a 10× objective lens. Representative sections throughout the medial and lateral portion of side were chosen for histological analysis, resulting in 5 sections being imaged for both portions of the joint.

### Immunohistochemistry

Seven micrometer-thick representative sections were chosen for immunohistochemistry, using a standard protocol (35). Tissue sections were deparaffinized, rehydrated, and washed in sodium borohydride (Sigma-Aldrich; 480886). Sections were then incubated with hyaluronidase (Sigma-Aldrich; H3505) and blocked with 5% serum in 1% PBST. Tissue sections were treated with primary antibodies to mouse ALK1 (R&D; AF770), Phospho-SMAD1/5 (Cell Signaling Technology; 9516), Phospho-SMAD2 (Cell Signaling Technology; 3108), MMP13 (Abcam; ab260040), and COL10 (Abcam; ab260040) and incubated overnight. Sections were then treated with a dilution of secondary antibody goat anti-rabbit IgG (H+L) Cross-Adsorbed Secondary Antibody, Alexa Flour 488 (Invitrogen; A-11008) or donkey anti-goat IgG (H+L) Cross-Adsorbed Secondary Antibody, Alexa Flour 488 (Invitrogen; A11055) for 4 hours. Finally, sections were incubated with 0.1 ug/mL of DAPI for 10 minutes before being mounted with Fluoro-Gel (Electron Microscopy Sciences; 17985-31). Images were taken with the Nikon Eclipse Ti fluorescent microscope.

### Micro-CT

Dissected knee joints underwent Micro-CT analysis using the Skyscan 1172 in vivo μCT scanner (Bruker micro-CT, Kontich, Belgium) at 55 kV and 181 μA, with a resolution of 10 μm. The raw data was then translated into two-dimensional cross-sectional gray scale image slices using NRecon (Bruker microCT, Kontich, Belgium). Structural parameters were then acquired from the two-dimensional images using a CT Analyzer (CT-AN, v1.10.9.0, Bruker microCT, Kontich, Belgium), including bone volume fraction (BV, mm3). The total knee osteophyte volume from the end of the distal femur to the end of the proximal tibia was compared between knees that underwent sham surgery, DMM surgery treated with PBS, DMM surgery treated with BMP-KI, or DMM surgery treated with ALK1-Fc. Average osteophyte volume was analyzed with Prism 9 Software (GraphPad) using two-way ANOVA and Tukey's multiple comparisons test post-hoc. MicroCT and data analysis was performed by an experienced investigator blinded to genotype/treatment.

### OARSI Scoring

The 10 total sections per joint chosen for histology received randomized sample numbers and were given to three independent researchers who scored the level of cartilage damage based on OARSI guidelines (84). OARSI scores were analyzed with Prism 9 Software (GraphPad) using two-way ANOVA and Tukey's multiple comparisons test post-hoc. Scoring was performed by at least three experienced investigators blinded to treatment/genotype. Scores were averaged for the medial tibial plateau, medial femoral condyle, lateral tibial plateau, and lateral femoral condyle for each sample.

### Subchondral Bone Analysis

For each experimental group, five joint sections per animal were analyzed from the medial compartment. The subchondral bone of the medial tibia was manually outlined while avoiding the articular cartilage and growth plate. The area of the outlined subchondral bone was divided by the approximate length of the subchondral bone to obtain the relative thickness of the subchondral bone based on previously described guidelines(85). The subchondral bone thickness across five representative sections were averaged for each animal. All quantifications were conducted in Fiji is Just ImageJ (FIJI, version 2.9.0). Quantified subchondral bone thickness was analyzed with Prism 9 Software (GraphPad) using two-way ANOVA and Tukey's multiple comparisons test post-hoc.

### Single-Cell RNA-Seq Analysis

Dataset was obtained from Chou et. al. using chondrocytes from articular cartilage obtained during joint replacement surgery of osteoarthritic patients (49). Initially, scRNA-seq analyses were validated with previous findings (FIG. 8A, B). Cluster analyses were confirmed to ensure that the cell types identified were consistent with the cell types identified by Chou et al who used the same data to identify chondrocyte subtypes(49). After successfully identifying chondrocyte subtypes, the expression of significant genes within the BMP and TGFbeta signaling pathways was examined to determine their expression within chondrocyte subtypes and to compare the difference between damaged OA and intact control chondrocytes. All analyses were conducted using R (version 4.2.0) in RStudio (version 2022 Feb. 3). The Seurat package was employed for data preprocessing and cluster analyses. Plots of scRNA-seq were visualized using t-SNE projection and violin plot.

### Pain Assessment.

Pain was assessed using the 50% paw withdrawal threshold (PWT) using Von Frey filaments and the up-and-down method as described(86, 87). Each mouse was tested three times by evaluators blinded to treatment group, and the average value was calculated. Whether or not each of the three acquired values is normal is based on the Jarque-Baera test. 4-5 mice were analyzed per treatment condition. Significance was assessed using two-way ANOVA and Tuckey's multiple comparisons test.

All procedures involving mice were approved by the Institutional Animal Care and Use Committee of University of California, Los Angeles. Mice were housed in an Association for Assessment and Accreditation of Laboratory Animal Care, an accredited facility in accordance with the Guide for the Care and Use of Laboratory Animals. This study was compliant with all relevant ethical regulations regarding animal research.

### Statistics

Statistics were performed with Prism 9 Software (GraphPad). Significance was assessed using one-way or two-way ANOVA and Tuckey's multiple comparisons post-hoc test.

### Example 1 References

1. Lo J, Chan L, Flynn S. A Systematic Review of the Incidence, Prevalence, Costs, and Activity and Work Limitations of Amputation, Osteoarthritis, Rheumatoid Arthritis, Back Pain, Multiple Sclerosis, Spinal Cord Injury, Stroke, and Traumatic Brain Injury in the United States: A 2019 Update. Arch Phys Med Rehabil. 2021; 102(1):115-131.
2. Kraan P M van der, Berg W B van den. Chondrocyte hypertrophy and osteoarthritis: role in initiation and progression of cartilage degeneration? Osteoarthritis and Cartilage. 2012; 20(3):223-232.
3. Ji Q, et al. Single-cell RNA-seq analysis reveals the progression of human osteoarthritis. Ann Rheum Dis. 2019; 78(1):100-110.
4. Laufer S. Osteoarthritis therapy—are there still unmet needs? Rheumatology (Oxford). 2004; 43 Suppl 1:i9-15.
5. Sharma L. Osteoarthritis of the Knee. N Engl J Med. 2021; 384(1):51-59.
6. van der Kraan P M, van den Berg W B. Osteophytes: relevance and biology. Osteoarthritis Cartilage. 2007; 15(3):237-244.
7. Roelofs A J, et al. Identification of the skeletal progenitor cells forming osteophytes in osteoarthritis. Ann Rheum Dis. 2020; 79(12):1625-1634.
8. Wang W, Rigueur D, Lyons K M. TGFβ Signaling in Cartilage Development and Maintenance. Birth Defects Res C Embryo Today. 2014; 102(1):37-51.
9. Boer C G, et al. Deciphering osteoarthritis genetics across 826,690 individuals from 9 populations. Cell. 2021; 184(18):4784-4818.e17.
10. MacFarlane E G, et al. TGF-β Family Signaling in Connective Tissue and Skeletal Diseases. Cold Spring Harb Perspect Biol. 2017; 9(11):a022269.
11. van der Kraan P M. Differential Role of Transforming Growth Factor-beta in an Osteoarthritic or a Healthy Joint. J Bone Metab. 2018; 25(2):65-72.
12. Shen J, Li S, Chen D. TGF-β signaling and the development of osteoarthritis. Bone Res. 2014; 2:14002.
13. Wang W, Rigueur D, Lyons K M. TGFβ as a gatekeeper of BMP action in the developing growth plate. Bone. 2020; 137:115439.
14. Yang X, et al. TGF-β/Smad3 Signals Repress Chondrocyte Hypertrophic Differentiation and Are Required for Maintaining Articular Cartilage. J Cell Biol. 2001; 153(1):35-46.
15. Chen C G, et al. Chondrocyte-intrinsic Smad3 represses Runx2-inducible matrix metalloproteinase 13 expression to maintain articular cartilage and prevent osteoarthritis. Arthritis Rheum. 2012; 64(10):3278-3289.
16. Shen J, et al. Deletion of the transforming growth factor β receptor type II gene in articular chondrocytes leads to a progressive osteoarthritis-like phenotype in mice. Arthritis Rheum. 2013; 65(12):3107-3119.
17. Chen R, et al. Attenuation of the progression of articular cartilage degeneration by inhibition of TGF-β1 signaling in a mouse model of osteoarthritis. Am J Pathol. 2015; 185(11):2875-2885.
18. Wang Q, et al. Cartilage-specific deletion of Alk5 gene results in a progressive osteoarthritis-like phenotype in mice. Osteoarthritis Cartilage. 2017; 25(11):1868-1879.
19. Rountree R B, et al. BMP receptor signaling is required for postnatal maintenance of articular cartilage. PLoS Biol. 2004; 2(11):e355.
20. Yoon B S, et al. Bmpr1a and Bmpr1b have overlapping functions and are essential for chondrogenesis in vivo. Proc Natl Acad Sci USA. 2005; 102(14):5062-5067.
21. Rigueur D, et al. The type I BMP receptor ACVR1/ALK2 is required for chondrogenesis during development. J Bone Miner Res. 2015; 30(4):733-741.
22. Salazar V S, Gamer L W, Rosen V. BMP signalling in skeletal development, disease and repair. Nat Rev Endocrinol. 2016; 12(4):203-221.
23. Jaswal A P, Bandyopadhyay A. Re-examining osteoarthritis therapy from a developmental biologist's perspective. Biochem Pharmacol. 2019; 165:17-23.
24. Jaswal A P, et al. BMP signaling: A significant player and therapeutic target for osteoarthritis. Osteoarthritis and Cartilage. [published online ahead of print: Jun. 29, 2023].
25. van der Kraan P M, Blaney Davidson E N, van den Berg W B. A role for age-related changes in TGFbeta signaling in aberrant chondrocyte differentiation and osteoarthritis. Arthritis Res Ther. 2010; 12(1):201.
26. Blaney Davidson E N, van der Kraan P M, van den Berg W B. TGF-beta and osteoarthritis. Osteoarthritis Cartilage. 2007; 15(6):597-604.
27. Whitty C, et al. Sustained delivery of the bone morphogenetic proteins BMP-2 and BMP-7 for cartilage repair and regeneration in osteoarthritis. Osteoarthr Cartil Open. 2022; 4(1):100240.
28. Chien S-Y, et al. Noggin Inhibits IL-1β and BMP-2 Expression, and Attenuates Cartilage Degeneration and Subchondral Bone Destruction in Experimental Osteoarthritis. Cells. 2020; 9(4):927.
29. Chawla S, et al. Blockage of bone morphogenetic protein signalling counteracts hypertrophy in a human osteoarthritic micro-cartilage model. J Cell Sci. 2020; 133(23):jcs249094.
30. Liu W, et al. Human osteoarthritis cartilage-derived stromal cells activate joint degeneration through TGF-beta lateral signaling. The FASEB Journal. 2020; 34(12):16552-16566.
31. Franco R A G, et al. Inhibition of BMP signaling with LDN 193189 can influence bone marrow stromal cell fate but does not prevent hypertrophy during chondrogenesis. Stem Cell Reports. 2022; 17(3):616-632.
32. Blaney Davidson E N, et al. Increase in ALK1/ALK5 ratio as a cause for elevated MMP-13 expression in osteoarthritis in humans and mice. J Immunol. 2009; 182(12):7937-7945.
33. Garcia de Vinuesa A, et al. Cripto favors chondrocyte hypertrophy via TGF-β SMAD1/5 signaling during development of osteoarthritis. J Pathol. 2021; 255(3):330-342.
34. van der Kraan P M. The changing role of TGFβ in healthy, ageing and osteoarthritic joints. Nat Rev Rheumatol. 2017; 13(3):155-163.
35. Wang W, et al. The TGFβ type I receptor TGFβRI functions as an inhibitor of BMP signaling in cartilage. Proceedings of the National Academy of Sciences. 2019; 116(31):15570-15579.
36. Townson S A, et al. Specificity and structure of a high affinity activin receptor-like kinase 1 (ALK1) signaling complex. J Biol Chem. 2012; 287(33):27313-27325.
37. Mohedas A H, et al. Structure-activity relationship of 3,5-diaryl-2-aminopyridine ALK2 inhibitors reveals unaltered binding affinity for fibrodysplasia ossificans progressiva causing mutants. J Med Chem. 2014; 57(19):7900-7915.
38. Dinter T, Bocobo G A, Yu P B. Pharmacologic Strategies for Assaying BMP Signaling Function. Methods Mol Biol. 2019; 1891:221-233.
39. Carvalho D, et al. ALK2 inhibitors display beneficial effects in preclinical models of ACVR1 mutant diffuse intrinsic pontine glioma. Commun Biol. 2019; 2:156.
40. Hawinkels L J, Garcia de Vinuesa A, Ten Dijke P. Activin receptor-like kinase 1 as a target for anti-angiogenesis therapy. Expert Opin Investig Drugs. 2013; 22(11):1371-1383.
41. David L, et al. Identification of BMP9 and BMP10 as functional activators of the orphan activin receptor-like kinase 1 (ALK1) in endothelial cells. Blood. 2007; 109(5):1953-1961.
42. Luo J, et al. TGFβ/BMP Type I Receptors ALK1 and ALK2 Are Essential for BMP9-induced Osteogenic Signaling in Mesenchymal Stem Cells. J Biol Chem. 2010; 285(38):29588-29598.
43. Astrologo L, et al. ALK1Fc Suppresses the Human Prostate Cancer Growth in in Vitro and in Vivo Preclinical Models. Front Cell Dev Biol. 2017; 5:104.
44. DiDomenico C D, et al. The Effect of Antibody Size and Mechanical Loading on Solute Diffusion Through the Articular Surface of Cartilage. J Biomech Eng. 2017; 139(9).
45. DiDomenico C D, Lintz M, Bonassar L J. Molecular transport in articular cartilage—what have we learned from the past 50 years? Nat Rev Rheumatol. 2018; 14(7):393-403.
46. Desroches-Castan A, et al. BMP9 and BMP10: Two close vascular quiescence partners that stand out. Dev Dyn. 2022; 251(1):178-197.
47. Voss M H, et al. A phase 2, randomized trial evaluating the combination of dalantercept plus axitinib in patients with advanced clear cell renal cell carcinoma. Cancer. 2019; 125(14):2400-2408.
48. Glasson S S, Blanchet T J, Morris E A. The surgical destabilization of the medial meniscus (DMM) model of osteoarthritis in the 129/SvEv mouse. Osteoarthritis Cartilage. 2007; 15(9):1061-1069.
49. Chou C-H, et al. Synovial cell cross-talk with cartilage plays a major role in the pathogenesis of osteoarthritis. Sci Rep. 2020; 10(1):10868.
50. Tetsunaga T, et al. Regulation of mechanical stress-induced MMP-13 and ADAMTS-5 expression by RUNX-2 transcriptional factor in SW1353 chondrocyte-like cells. Osteoarthritis Cartilage. 2011; 19(2):222-232.
51. Ovchinnikov D A, et al. Col2a1-directed expression of Cre recombinase in differentiating chondrocytes in transgenic mice. Genesis. 2000; 26(2):145-146.
52. Mitchell D, et al. ALK1-Fc inhibits multiple mediators of angiogenesis and suppresses tumor growth. Mol Cancer Ther. 2010; 9(2):379-388.
53. Venne G, et al. Mechanically-induced osteophyte in the rat knee. Osteoarthritis Cartilage. 2020; 28(6):853-864.
54. Deuis J R, Dvorakova L S, Vetter I. Methods Used to Evaluate Pain Behaviors in Rodents. Front Mol Neurosci. 2017; 10:284.
55. Glyn-Jones S, et al. Osteoarthritis. Lancet. 2015; 386(9991):376-387.
56. Sharma L. Osteoarthritis of the Knee. N Engl J Med. 2021; 384(1):51-59.
57. Roseti L, et al. Articular Cartilage Regeneration in Osteoarthritis. Cells. 2019; 8(11):1305.
58. Thielen N G M, van der Kraan P M, van Caam A P M. TGFβ/BMP Signaling Pathway in Cartilage Homeostasis. Cells. 2019; 8(9):969.
59. Wu M, Chen G, Li Y-P. TGF-β and BMP signaling in osteoblast, skeletal development, and bone formation, homeostasis and disease. Bone Res. 2016; 4(1):1-21.
60. Keller B, et al. Interaction of TGFβ and BMP Signaling Pathways during Chondrogenesis. PLOS ONE. 2011; 6(1):e16421.
61. Tan Q, et al. TGF-β/Alk5 signaling prevents osteoarthritis initiation via regulating the senescence of articular cartilage stem cells. J Cell Physiol. 2021; 236(7):5278-5292.
62. Voss M H, et al. A phase 2, randomized trial evaluating the combination of dalantercept plus axitinib in patients with advanced clear cell renal cell carcinoma. Cancer. 2019; 125(14):2400-2408.
63. Sanvitale C E, et al. A new class of small molecule inhibitor of BMP signaling. PLoS One. 2013; 8(4):e62721.
64. Ruan X, et al. Multiple roles of ALK3 in osteoarthritis. Bone Joint Res. 2023; 12(7):397-411.
65. van Caam A, et al. Expression of TGFβ-family signalling components in ageing cartilage: age-related loss of TGFβ and BMP receptors. Osteoarthritis Cartilage. 2016; 24(7):1235-1245.
66. Jing Y, et al. Chondrogenesis and osteogenesis are one continuous developmental and lineage defined biological process. Sci Rep. 2017; 7(1):10020.
67. Schmal H, et al. Expression of BMP-receptor type 1A correlates with progress of osteoarthritis in human knee joints with focal cartilage lesions. Cytotherapy. 2012; 14(7):868-876.
68. Dreier R. Hypertrophic differentiation of chondrocytes in osteoarthritis: the developmental aspect of degenerative joint disorders. Arthritis Research & Therapy. 2010; 12(5):216.
69. Salmon R M, et al. Molecular basis of ALK1-mediated signalling by BMP9/BMP10 and their prodomain-bound forms. Nat Commun. 2020; 11:1621.
70. Lauwers M, et al. The cholinergic system in joint health and osteoarthritis: a narrative-review. Osteoarthritis Cartilage. 2021; 29(5):643-653.
71. López-Coviella I, et al. Induction and maintenance of the neuronal cholinergic phenotype in the central nervous system by BMP-9. Science. 2000; 289(5477):313-316.
72. Song B, et al. BMP9 inhibits the proliferation and migration of fibroblast-like synoviocytes in rheumatoid arthritis via the PI3K/AKT signaling pathway. Int Immunopharmacol. 2019; 74:105685.
73. Ulmner M, et al. Synovial Tissue Proteins and Patient-Specific Variables as Predictive Factors for Temporomandibular Joint Surgery. Diagnostics (Basel). 2020; 11(1):46.
74. Eckstein F, et al. CARTILAGE THICKNESS CHANGE AS AN IMAGING BIOMARKER OF KNEE OSTEOARTHRITIS PROGRESSION—DATA FROM THE FNIH OA BIOMARKERS CONSORTIUM. Arthritis Rheumatol. 2015; 67(12):3184-3189.
75. Sun Q, et al. Peripheral nerves in the tibial subchondral bone. Bone Joint Res. 2022; 11(7):439-452.
76. Suri S, et al. Neurovascular invasion at the osteochondral junction and in osteophytes in osteoarthritis. Ann Rheum Dis. 2007; 66(11):1423-1428.
77. Fan T, et al. Osteophytes mediate the associations between cartilage morphology and changes in knee symptoms in patients with knee osteoarthritis. Arthritis Res Ther. 2022; 24:217.
78. Kang Q, et al. A Comprehensive Analysis of the Dual Roles of BMPs in Regulating Adipogenic and Osteogenic Differentiation of Mesenchymal Progenitor Cells. Stem Cells Dev. 2009; 18(4):545-558.
79. Mapp P I, Walsh D A. Mechanisms and targets of angiogenesis and nerve growth in osteoarthritis. Nat Rev Rheumatol. 2012; 8(7):390-398.
80. Donell S. Subchondral bone remodelling in osteoarthritis. EFORT Open Rev. 2019; 4(6):221-229.
81. Scanzello C R, Goldring S R. The role of synovitis in osteoarthritis pathogenesis. Bone. 2012; 51(2):249-257.
82. Ge Q, et al. Protein phosphatase PPM1A inhibition attenuates osteoarthritis via regulating TGF-β/Smad2 signaling in chondrocytes. JCI Insight. 2023; 8(3).
83. Zhen G, et al. Inhibition of TGF-β signaling in mesenchymal stem cells of subchondral bone attenuates osteoarthritis. Nat Med. 2013; 19(6):704-712.
84. Glasson S S, et al. The OARSI histopathology initiative—recommendations for histological assessments of osteoarthritis in the mouse. Osteoarthritis Cartilage. 2010; 18 Suppl 3:S17-23.
85. Nagira K, et al. Histological scoring system for subchondral bone changes in murine models of joint aging and osteoarthritis. Sci Rep. 2020; 10(1):10077.
86. Chaplan S R, et al. Quantitative assessment of tactile allodynia in the rat paw. J Neurosci Methods. 1994; 53(1):55-63.
87. Wang J, et al. A Single Sub-anesthetic Dose of Ketamine Relieves Depression-like Behaviors Induced by Neuropathic Pain in Rats. Anesthesiology. 2011; 115(4):812-821.

All publications mentioned herein (e.g., U.S. Pat. No. 8,158,584 and the publications numerically listed above) are incorporated herein by reference to disclose and describe aspects, methods and/or materials in connection with the cited publications.

1. A method of promoting cartilage preservation, repair, and/or regeneration in a mammal, the method comprising administering to the mammal a therapeutically effective amount of at least one agent selected from ALK1-Fc, a growth differentiation factor 11 (GDF11) polypeptide and LDN-214117, such that cartilage preservation, repair, and/or regeneration is promoted.

2. The method of claim 1, wherein the mammal is selected to be a human diagnosed with osteoarthritis (OA).

3. The method of claim 1, wherein the mammal is administered an amount of agent selected to be sufficient to inhibit heterotopic ossification, osteophyte formation, and/or destruction of articular cartilage in vivo.

4. The method of claim 1, wherein the mammal is administered the agent intra-articularly.

5. A method of mitigating patient pain associated with damage to articular cartilage, the method comprising combining the articular cartilage with amounts of at least one agent selected from ALK1-Fc and LDN-214117 selected to be sufficient to:
inhibit pain in the patient following trauma to the articular cartilage; and
inhibit degradation of the articular cartilage following trauma to the articular cartilage; or
inhibit development and/or progression of osteoarthritis following trauma to the articular cartilage;
such that pain associated with damage to articular cartilage is mitigated.

6. The method of claim 5, wherein the articular cartilage is combined with the agent by intra-articular injection of the agent.

7. The method of claim 5, wherein the articular cartilage is disposed in a human diagnosed with osteoarthritis (OA).

8. The method of claim 5, wherein ALK1-Fc and LDN-214117 are administered to the patient.

9. The method of claim 5, wherein ALK1-Fc is administered by intra-articular injection in amounts of 300 ng-500 ng per dose per week for at least 1-8 weeks

10. A method of modulating the physiology of an articular cartilage, the method comprising combining the articular cartilage with amounts of at least one agent selected from ALK1-Fc, a growth differentiation factor 11 (GDF11) polypeptide and LDN-214117 selected to be sufficient to:
inhibit fibrocartilage formation;
inhibit osteophyte formation;
thereby modulating the physiology of articular cartilage.

11. A composition of matter comprising at least one agent selected from ALK1-Fc, a growth differentiation factor 11 (GDF11) polypeptide and LDN-214117, wherein amounts of the agent in the compositions is sufficient to inhibit development and/or progression of osteoarthritis in a patient following trauma to the articular cartilage when the composition is disposed in the patient.

12. The composition of claim 1, wherein the composition comprises at least two agents.

13. The composition of claim 1, wherein the composition comprises ALK1-Fc.

14. The composition of claim 1, wherein the composition comprises a growth differentiation factor 11 (GDF11) polypeptide.

15. The composition of claim 1, wherein the composition comprises LDN-214117.
